(19) United States
(12) Patent Application Publication
(10) Pub. No.: US 2021/0228713 A1
(43) Pub. Date: Jul. 29, 2021

(54) TLR7 PEPTIDE CONJUGATES

(71) Applicant: Apros Therapeutics, Inc., San Diego, CA (US)

(72) Inventor: Tom Yao-Hsiang WU, San Diego, CA (US)

(21) Appl. No.: 15/734,922

(22) PCT Filed: Jun. 3, 2019
(86) PCT No.: PCT/US2019/035194

§ 371 Date: Dec. 3, 2020

Related U.S. Application Data
(60) Provisional application No. 62/680,332, filed on Jun. 4, 2018.

(51) Int. Cl. A61K 39/39; A61P 35/00; A61K 47/64; A61K 39/00

(52) U.S. Cl. CPC A61K 39/39; A61K 2039/55511; A61K 47/646; A61P 35/00

(57) ABSTRACT

The present disclosure relates to a class of pyrimidine derivative peptide conjugates having enhanced immunomodulating properties. More specifically the peptide conjugate contains a TLR7 agonist and enhances the biological effect of the peptide to which it is coupled, increasing immunogenicity, and or lowering the effective dose of the peptide. In some embodiments, the peptide is an antigen, a vaccine, a peptide-based neoantigen vaccine, or an epitope.

### CROSS REFERENCE TO RELATED APPLICATIONS

This application claims the benefit of U.S. Provisional Application No. 62/680,332, filed Jun. 4, 2018, the contents of which are incorporated herein by reference in its entirety.

### FIELD OF THE INVENTION

The present disclosure relates to a class of pyrimidine derivative peptide conjugates having enhanced immunomodulating properties.

### BACKGROUND OF THE INVENTION

Different vaccine platforms can target distinct arms of the immune system with varying degrees of potency. For example, viral/nucleic acid vaccines (Vectors) are highly effective at driving CD8+ (Killer) T cell responses while subunit vaccines are poor inducers of killer T cells, but strong inducers of antibody and CD4+ (Helper) T cell responses (D'Argenio and Wilson. Immunity. 2010. 33:437-40). Peptide vaccines are a platform routinely used to provide defined epitopes for presentation to helper and/or killer T cells by the major histocompatibility (MHC) molecules of antigen presenting cells (APC). Peptide vaccines can also be used to induce antibody responses against linear epitopes recognized by B cells (Winblad et al. The Lancet. 2012. 11:597-604).

Compared to live-attenuated microorganisms, nucleic acid vectors and recombinant protein subunits, peptide vaccines have the advantages of defined chemical structure and rapid manufacture. However, peptide vaccines are often poorly immunogenic due to their short half-life after injection and therefore, require an adjuvant to achieve optimal vaccine potency. As a result, most vaccines are comprised of antigen(s) and adjuvant, whereby the antigen provides the specific target for T and/or B cell receptors and the adjuvant component serves as a general immune enhancer of antigen-specific responses (O'Hagan and Valiante Nat Rev Drug Discov. 2003. 9:727-35).

Peptide vaccines have been most extensively studied in the context of prophylaxis and therapy for viral infections and cancer, where killer and helper T cell responses are critical for host defense against these diseases. Recently, an important class of T cell antigens (tumor neoantigens) have been identified that can be readily targeted with peptide vaccines. Next-generation sequencing (NGS) and bioinformatics are used to identify tumor mutations that have the potential to be recognized as neoantigens. Due to the patient-specific nature of neoantigens, rapid synthesis of the epitopes is required for the development of personalized cancer vaccines. As such, peptide-based neoantigen vaccines have received considerable attention as the basis of new cancer therapies. Scientific articles have described a personalized neoantigen vaccine for use patients with melanoma, and shown that the vaccine was immunogenic in humans (Wu et. al. Nature, 2017, 547, 217-221). In that vaccine, poly-IC (a TLR3 agonist) was used as the adjuvant by admixing with the synthetic peptide antigens.

However, coadministering a peptide antigen and adjuvant that are non-covalently attached may result in separation post-injection. The peptide antigen and adjuvant may concentrate in different tissues and the immunogenicity-enhancing benefits of the adjuvant diminished. This results in reduced efficacy of the peptide antigen. There is a need for compounds and methods that enhance the antigen-specific responses of a vaccine and improve the benefits of a coadministered adjuvant.

### SUMMARY OF THE INVENTION

In one aspect, the disclosure describes a peptide antigen covalently conjugated to a synthetic TLR7 agonist. In another aspect, the peptide is synthesized using conventional solid-phase peptide synthesis and is coupled with a TLR7 agonist at the last step prior to resin cleavage. This process adds minimal complexity to the current peptide synthesis and provides a sequence modified with a TLR7 agonist at the N-terminus. The peptide conjugates described herein increase the immunogenicity of the peptide antigens and/or lower the effective doses. In a vaccine that comprises multiple peptide antigens, an immunogenicity-enhancing and/or dosing-sparing adjuvant technology can greatly improve the overall efficacy, convenience, and cost-effectiveness of the vaccine. Typically the peptide conjugate is prepared by forming a covalent bond between the TLR agonist and the peptide, such as an antigen, a vaccine, a peptide-based neoantigen vaccine, or an epitope.

In one aspect, the disclosure provides a peptide conjugate having the structure of Formula I, or a pharmaceutically acceptable salt thereof,

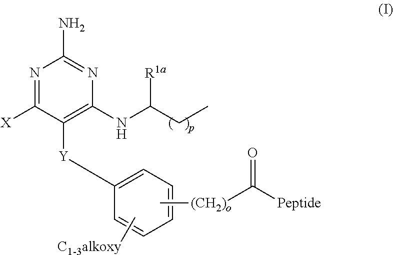

wherein

Peptide is a peptide, wherein (C═O) is attached to (i) the N-terminus of the peptide, or (ii) a side chain of the peptide where the functional group of the side chain to which C═O is attached is NH₂;

R^{1a} is H or C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

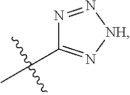 [SMILES: *c1nn[nH]n1]

### and A^{l};

X is H or C₁-C₄ alkyl, wherein the alkyl is optionally substituted with one to three substituents selected from the group consisting of A², —OH, and —C(CH₃)₂OH;

Y is selected from the group consisting of a bond, —CH₂—, —CF₂—,

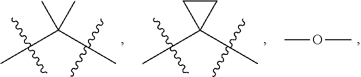

—S—, —SO₂—, —NH—, and —CH₂CH₂—;

A^{l} is selected from the group consisting of

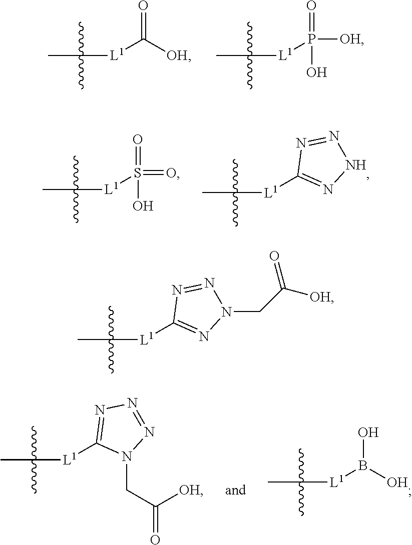

L¹ is selected from the group consisting of a bond, —(CH₂)ₙ—, —C(O)NH(CH₂)ₙ—,

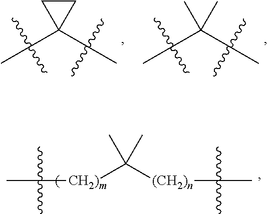

—[O(CH₂CH₂)]ₙ, —[O(C₁-C₄ alkylene)]-, —[O(CH₂CH₂)]ₙ-—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

A² is selected from the group consisting of

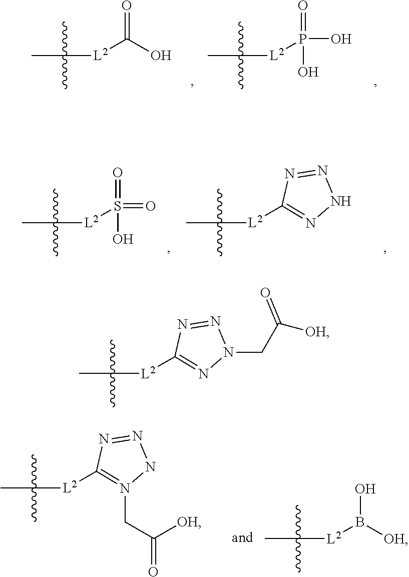

L² is selected from the group consisting of a bond, —(CH₂)ₙ—, —C(O)NH(CH₂)ₙ—,

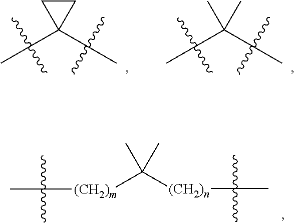

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

m is an integer from zero to four;

n and p are independently an integer from one to four; and

o is an integer from zero to four.

In one aspect, the disclosure provides a peptide conjugate as described herein, or a pharmaceutically acceptable salt thereof, wherein the peptide is prepared by solid phase synthesis.

In another aspect, the disclosure provides a pharmaceutical composition comprising the peptide conjugate as described herein, or a pharmaceutically acceptable salt thereof.

In another aspect, the disclosure provides a method of making a conjugate as described herein, or a pharmaceutically acceptable salt thereof, comprising the step of conjugating a peptide with a compound of Formula (3a)

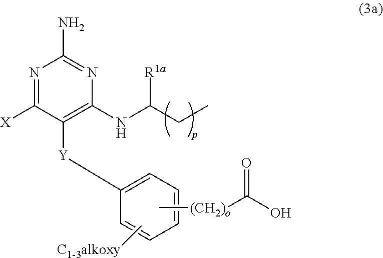

wherein

R^{1a} is H or C₁-C₄ alkyl, wherein the alkyl is optionally substituted with one to three substituents selected from the group consisting of —OZ, —NHZ, —NHAc, —COOZ, —SO₂CH₃, —SCH₃, —OCH₃,

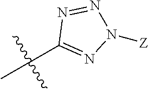

### and A¹;

X is H or C₁-C₄ alkyl, wherein the alkyl is optionally substituted with one to three substituents selected from the group consisting of A², —OZ, and —C(CH₃)₂OZ;

Y is selected from the group consisting of a bond, —CH₂—, —CF₂—,

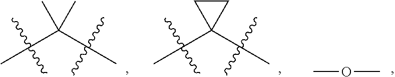

—S—, —SO₂—, —NH—, and —CH₂CH₂—;

A¹ is selected from the group consisting of

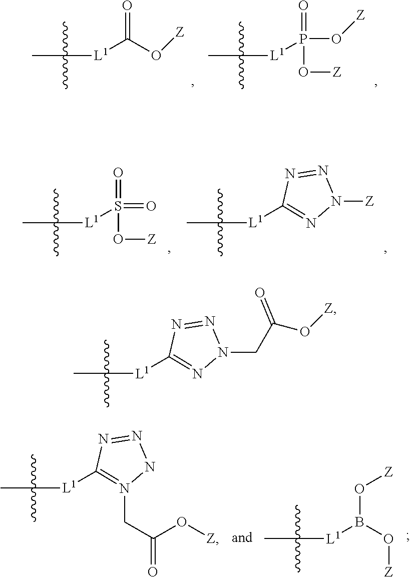

L¹ is selected from the group consisting of a bond, —(CH₂)ₙ—, —C(O)NH(CH₂)ₙ—,

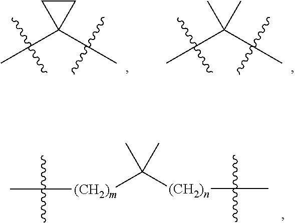

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

A² is selected from the group consisting of

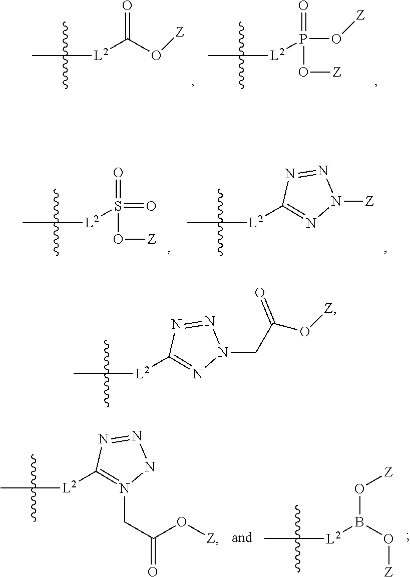

L² is selected from the group consisting of a bond, —(CH₂)n—, —C(O)NH(CH₂)ₙ—,

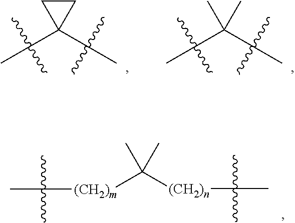

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

each Z is independently H or a protecting group;

m is an integer from zero to four;

n and p are independently an integer from one to four; and

o is an integer from zero to four.

In another aspect, the disclosure provides a peptide conjugate as described herein, or a pharmaceutically acceptable salt thereof, for use in therapy.

In another aspect, the disclosure provides a pharmaceutical composition as described herein, for use in therapy.

In another aspect, the disclosure provides for the use of a peptide conjugate as described herein, or a pharmaceutically acceptable salt thereof, in the manufacture of a medicament.

### DETAILED DESCRIPTION

Although specific embodiments of the present disclosure are herein illustrated and described in detail, the invention is not limited thereto. The detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

### Terms

The following definitions are meant to clarify, but not limit, the terms defined. If a particular term used herein is not specifically defined, such term should not be considered indefinite. Rather, terms are used within their accepted meanings.

The term “alkyl” as used herein refers to a straight or branched saturated hydrocarbon. For example, an alkyl group can have 1 to 8 carbon atoms (i.e., (C₁-C₈)alkyl) or 1 to 6 carbon atoms (i.e., (C₁-C₆)alkyl) or 1 to 4 carbon atoms (i.e., (C₁-C₄)alkyl).

The term “alkylene” as used herein refers to a straight or branched saturated hydrocarbon radical having two monovalent radical centers derived by the removal of two hydrogen atoms from the same or two different carbon atoms of a parent alkane. For example, an alkylene group can have 1 to 8 carbon atoms, 1 to 6 carbon atoms, or 1 to 4 carbon atoms.

The term “alkoxy” as used herein refers to an alkyl group attached to the parent molecular moiety through an oxygen atom.

The term “peptide” as used herein refers to a compound comprised of two or more amino acids units covalently linked by peptide bonds. Dipeptides have two amino acid residues, tripeptides have three, tetrapeptides have four, and so on. Peptides may include oligopeptides, polypeptides, and proteins.

The term “amino acid” as used herein refers to natural and synthetic amino acids, and both D and L amino acids. “Natural amino acid” means any of the twenty primary, naturally occurring amino acids which typically form peptides, polypeptides, and proteins. “Synthetic amino acid” means any other amino acid, regardless of whether it is prepared synthetically or derived from a natural source. As used herein, “synthetic amino acid” also encompasses chemically modified amino acids, including but not limited to salts, derivatives (such as amides), and substitutions. Amino acids contained within the peptides of the present disclosure, and particularly at the carboxy- or amino-terminus, can be modified by methylation, amidation, acetylation or substitution with other chemical groups. Additionally, a disulfide linkage may be present or absent in the peptides of the disclosure.

The term “amino acid residue” as used herein refers to an amino acid unit in the peptide.

The term “residue” as used herein refers to what is left after the release of H₂O when an amino acid forms a peptide link upon joining the peptide chain.

The term “oligopeptide” as used herein refers to peptide chains of more than 12 and less than about 20 amino acid residues.

The term “polypeptide” as used herein refers to a peptide chain of more than about 20 amino acid residues.

The term “protein” as used herein refers to molecules composed of one or more polypeptide chains.

As used herein, the term “pharmaceutically acceptable” refers to carrier(s), diluent(s), excipient(s) or salt forms that are compatible with the other ingredients of the formulation and not deleterious to the recipient of the pharmaceutical composition.

As used herein, the term “pharmaceutical composition” refers to a compound of the present disclosure optionally admixed with one or more pharmaceutically acceptable carriers, diluents, excipients, or adjuvants. Pharmaceutical compositions preferably exhibit a degree of stability to environmental conditions so as to make them suitable for manufacturing and commercialization purposes.

As used herein, the terms “effective amount,” “therapeutic amount,” or “effective dose” refer to an amount of active ingredient sufficient to elicit the desired pharmacological or therapeutic effects, thus resulting in effective prevention or treatment of a disorder. Prevention of a disorder may be manifested by delaying or preventing the progression of the disorder, as well as delaying or preventing the onset of the symptoms associated with the disorder. Treatment of the disorder may be manifested by a decrease or elimination of symptoms, inhibition or reversal of the progression of the disorder, as well as any other contribution to the well-being of the patient.

The effective dose can vary, depending upon factors such as the condition of the patient, the severity of the symptoms of the disorder, and the manner in which the pharmaceutical composition is administered. Typically, to be administered in an effective dose, compounds are required to be administered in an amount of less than 30 mg. Often, the compounds may be administered in an amount from less than about 1 mg to less than about 100 μg, and occasionally between about 10 μg to less than 100 μg. The foregoing effective doses typically represent that amount administered as a single dose, or as one or more doses administered over a 24 hours period. For human patients, the effective dose of the compounds may require administering the compound in an amount of at least about 1 μg/24 hr/patient, but not more than about 2400 μg/24 hr/patient, and often not more than about 500 μg/24 hr/patient.

Peptide Conjugate

In one aspect, the disclosure provides a peptide conjugate having the structure of Formula I, or a pharmaceutically acceptable salt thereof,

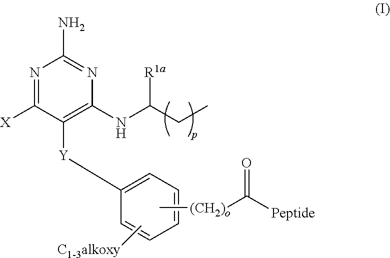

wherein

Peptide is a peptide, wherein (C═O) is attached to (i) the N-terminus of the peptide, or (ii) a side chain of the peptide where the functional group of the side chain to which C═O is attached is NH₂;

R^{1a} is H or C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

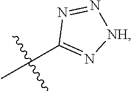 [SMILES: *c1nn[nH]n1]

### and A¹;

X is H or C₁-C₄ alkyl, wherein the alkyl is optionally substituted with one to three substituents selected from the group consisting of A², —OH, and —C(CH₃)₂OH;

Y is selected from the group consisting of a bond, —CH₂—, —CF₂—,

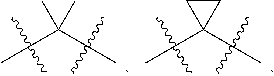

—O—, —S—, —SO₂—, —NH—, and —CH₂CH₂—;

A¹ is selected from the group consisting of

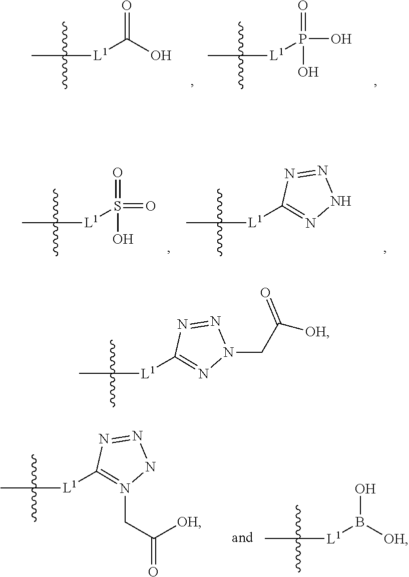

L¹ is selected from the group consisting of a bond, —(CH₂)ₙ—, —C(O)NH(CH₂)ₙ—,

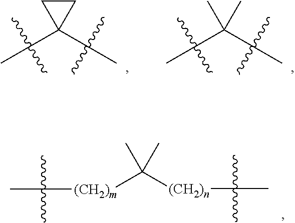

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —(O)NHCH₂CH₂—[O(CH₂CH₂)]ₘOCH₂CH₂CF₂—;

A² is selected from the group consisting of

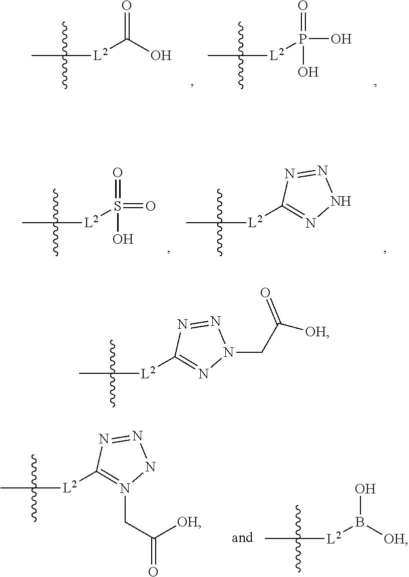

L² is selected from the group consisting of a bond, —(CH₂)ₙ—, —C(O)NH(CH₂)ₙ—,

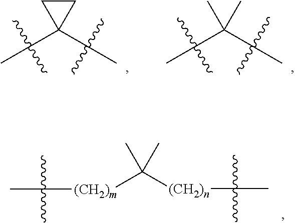

—[O(CH₂CH₂)]ₙ—, —[O(C₁-C₄ alkylene)]-, —[O(CH₂CH₂)]ₙ—OCH₂CH₂CF₂—, —C(O )NHCH₂CH₂—[O(CH₂CH₂)]ₘ—, and —C(O)NHCH₂CH₂—[O(CH₂CH₂)]ₘ—OCH2CH₂CF₂—;

m is an integer from zero to four;

n and p are independently an integer from one to four; and

o is an integer from zero to four.

In some embodiments, the present disclosure provides a peptide conjugate having the structure of Formula I, or a pharmaceutically acceptable salt thereof, wherein Formula I has the structure of Formula (1a) or (2a),

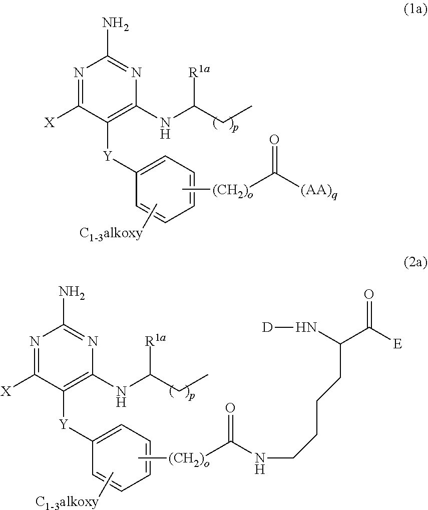

wherein
each AA is independently an amino acid, wherein the (AA)_{q} is a peptide, wherein (C═O) is attached to the N-terminus of the peptide;
q is an integer from eight to forty;
D is H or an amino acid or a peptide comprising 2 to 40 amino acids;
E is OH or an amino acid or a peptide comprising 2 to 40 amino acids; and
R^{1a}, X, Y, p, and o are as defined for Formula I.

In some embodiments, the present disclosure provides a peptide conjugate of Formula 1a, or a pharmaceutically acceptable salt thereof, wherein Formula 1a has the structure of Formula 1b

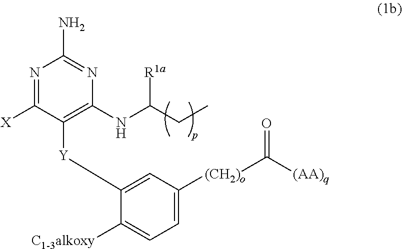

wherein AA and q are as defined for Formulas 1a and 2a; and
R^{1a}, X, Y, p, and o are as defined for Formula I.

In some embodiments, the present disclosure provides a peptide conjugate of Formula 2a, or a pharmaceutically acceptable salt thereof, wherein Formula 2a has the structure of Formula 2b

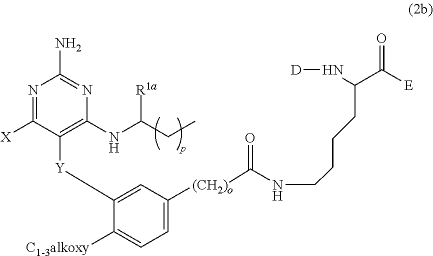

wherein D, and E are as defined for Formulas 1a and 2a; and
R^{1a}, X, Y, p, and o are as defined for Formula I.

In some embodiments, the disclosure provides a peptide conjugate of Formula I, Formula 1a, Formula 2a, Formula 1b, or Formula 2b, or a pharmaceutically acceptable salt thereof, wherein (C═O ) is attached to the N-terminus of the peptide. In alternative embodiments, (C═O) is attached to a side chain of the peptide where the functional group of the side chain to which C═O) is attached is NH₂.

In some embodiments, the disclosure provides a peptide conjugate of Formula I, Formula 1a, Formula 2a, Formula 1b, or Formula 2b, or a pharmaceutically acceptable salt thereof, wherein the amino acid is a natural amino acid. In some embodiments, the amino acid is a synthetic amino acid. In some embodiments, the amino acid is a natural or synthetic amino acid.

In some embodiments, the disclosure provides a peptide conjugate of Formula I, Formula 1a, Formula 2a, Formula 1b, or Formula 2b, or a pharmaceutically acceptable salt thereof, wherein R^{1a} is H. In alternative embodiments, R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

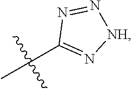 [SMILES: *c1nn[nH]n1]

and A¹. In some embodiments, R^{1a} is C₁-C₄ alkyl, wherein the alkyl is optionally substituted with —SO₂CH₃. In some embodiments, R^{1a} is C₁-C₄ alkyl, wherein the alkyl is substituted with —SO₂CH₃. In more specific embodiments, R^{1a} is

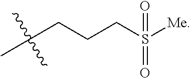 [SMILES: *CCCS(C)(=O)=O]

In some embodiments, the disclosure provides a peptide conjugate of Formula I, Formula 1a, Formula 2a, Formula 1b, or Formula 2b, or a pharmaceutically acceptable salt thereof, wherein X is H. In alternative embodiments, X is C₁-C₄ alkyl. In a specific embodiment, X is methyl. In some embodiments, X is C₁-C₄ alkyl, wherein the alkyl is optionally substituted with one to three substituents selected from the group consisting of A², —OH, and —C(CH₃)₂OH.

In some embodiments, the disclosure provides a peptide conjugate of Formula I, Formula 1a, Formula 2a, Formula 1b, or Formula 2b, or a pharmaceutically acceptable salt thereof, wherein Y is —CH₂—.

In some embodiments, the disclosure provides a peptide conjugate of Formula I, Formula 1a, Formula 2a, Formula 1b, or Formula 2b, or a pharmaceutically acceptable salt thereof, wherein o is zero. In some embodiments, o is one. In some embodiments, o is two. In some embodiments, o is three. In some embodiments, o is four.

In some embodiments, the disclosure provides a peptide conjugate of Formula I, Formula 1a, Formula 2a, Formula 1b, or Formula 2b, or a pharmaceutically acceptable salt thereof, wherein p is one. In some embodiments, p is two. In some embodiments, p is three.

In some embodiments, the disclosure provides a peptide conjugate of Formula I, Formula 1a, Formula 2a, Formula 1b, or Formula 2b, or a pharmaceutically acceptable salt thereof, wherein R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

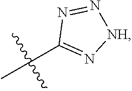 [SMILES: *c1nn[nH]n1]

### and A^{l};

X is C₁-C₄ alkyl; and

Y is —CH₂—.

In some embodiments, the disclosure provides a peptide conjugate of Formula I, Formula 1a, Formula 2a, Formula 1b, or Formula 2b, or a pharmaceutically acceptable salt thereof, having one, two, or three or more of the following features:

a) R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

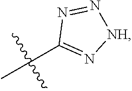 [SMILES: *c1nn[nH]n1]

### and A¹;

b) X is C₁-C₄ alkyl;

c) Y is —CH₂—;

d) C₁₋₃ alkoxy is CH₃O—;

e) o is one;

f) p is three; and

g) peptide is an antigen or vaccine.

In some embodiments, the disclosure provides a peptide conjugate of Formula I, Formula 1a, Formula 2a, Formula 1b, or Formula 2b, or a pharmaceutically acceptable salt thereof, having one, two, or three or more of the following features:

a) R^{1a} is C₁-C₄ alkyl, wherein the alkyl is substituted with —SO₂CH₃;

b) X is C₁-C₄alkyl;

c) Y is —CH₂—;

d) C₁₋₃ alkoxy is CH₃O—;

e) o is one;

f) p is three; and

g) peptide is an antigen or vaccine.

In some embodiments, the disclosure provides a peptide conjugate of Formula I, Formula 1a, Formula 2a, Formula 1b, or Formula 2b, or a pharmaceutically acceptable salt thereof, having one, two, or three or more of the following features:

a) R^{1a} is

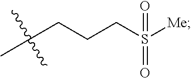 [SMILES: *CCCS(C)(=O)=O]

b) X is methyl;

c) Y is —CH₂—;

d) C₁₋₃ alkoxy is CH₃O—;

e) o is one;

f) p is three; and

g) peptide is an antigen or vaccine.

Peptides

The disclosure provides a peptide conjugate having the structure of Formula I, Formula 1a, Formula 2a, Formula 1b, or Formula 2b, or a pharmaceutically acceptable salt thereof, wherein Peptide is a peptide. In one embodiment, the peptide is an oligopeptide. In another embodiment, the peptide is a polypeptide. In yet another embodiment, the peptide is a protein.

The disclosure provides peptide conjugates having the structure of Formula I, Formula 1a, Formula 2a, Formula 1b, or Formula 2b, or a pharmaceutically acceptable salt thereof, wherein the peptide of the peptide conjugate is indicated below:

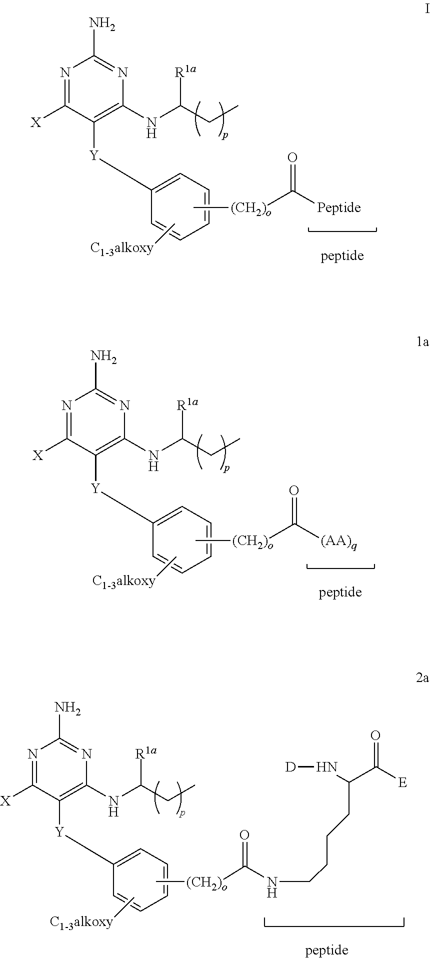

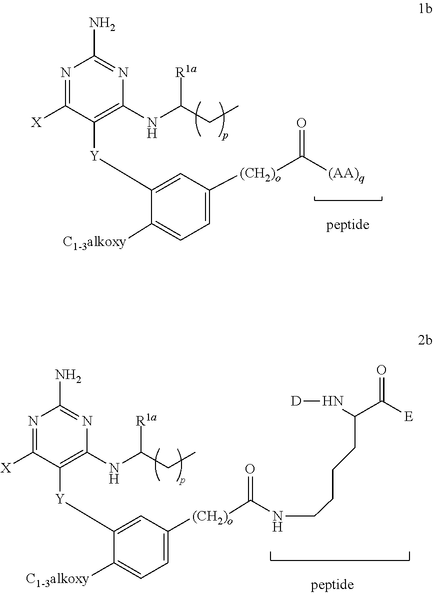

In certain embodiments, references made to ‘peptide’ as it relates to the peptide of the peptide conjugate, refers to the portion of the peptide conjugate shown in the table above.

In some embodiments, peptide is comprised of natural amino acids. In some embodiments, the peptide is comprised of synthetic amino acids. In some embodiments, the peptide is comprised of both natural and synthetic amino acids. In some embodiments, the peptide consists of natural amino acids. In some embodiments, the peptide consists of synthetic amino acids. In some embodiments, the peptide consists of both natural and synthetic amino acids.

In some embodiments, the disclosure provides a peptide conjugate of Formula I, Formula 1a, Formula 2a, Formula 1b, or Formula 2b, or a pharmaceutically acceptable salt thereof, wherein the peptide is an antigen. In some embodiments, the antigen is a bacterial or viral antigen. In some embodiments, the antigen is an epitope. In some embodiments, the antigen is a shared tumor antigen. In some embodiments, the antigen is a personalized neoantigen. In some embodiments, the peptide is a vaccine.

In some embodiments, the disclosure provides a peptide conjugate of Formula I, Formula 1a, Formula 2a, Formula 1b, or Formula 2b, or a pharmaceutically acceptable salt thereof, wherein the peptide is comprised of 2 to 80 amino acids, 2 to 40 amino acids, 2 to 20 amino acids or 2 to 10 amino acids. In some embodiments, the peptide is comprised of 10 to 80 amino acids, 10 to 40 amino acids, 10 to 30 amino acids or 10 to 25 amino acids. In some embodiments, the peptide is comprised of 20 to 80 amino acids, 20 to 40 amino acids, 20 to 30 amino acids or 20 to 25 amino acids.

In some embodiments, the disclosure provides a peptide conjugate of Formula 1a, Formula 1b, and pharmaceutically acceptable salts thereof, wherein q is an integer from 2 to 80; 2 to 40; 2 to 20; or 2 to 10. In some embodiments, q is an integer from 8 to 80; 8 to 40; 8 to 30; 8 to 20; or 8 to 10.

The disclosure provides a peptide conjugate of Formula 2a, Formula 2b, and pharmaceutically acceptable salts thereof, wherein D is H and E is 1 to 40 amino acids. In some embodiments, D is 1 to 40 amino acids and E is OH. In some embodiments, D is 1 to 20 amino acids and E is 1 to 20 amino acids. In some embodiments, D is 1 to 30 amino acids and E is 1 to 10 amino acids. In some embodiments, D is 1 to 10 amino acids and E is 1 to 30 amino acids.

Preparation of Peptide Conjugate

In one aspect, the disclosure provides a peptide conjugate as described herein, or a pharmaceutically acceptable salt thereof, wherein the peptide is prepared by solid phase synthesis. In one embodiment, the disclosure provides a peptide conjugate as described herein, or a pharmaceutically acceptable salt thereof, wherein formation of a covalent bond between the peptide and the TLR7 agonist happens when the peptide is bound to a solid phase. In some embodiments, formation of a covalent bond between the peptide and the TLR7 agonist happens when the peptide is bound to a solid phase and the resulting peptide conjugate is cleaved from the solid phase.

In another aspect, the disclosure provides a method of making a peptide conjugate as described herein, or a pharmaceutically acceptable salt thereof, comprising the step of conjugating a peptide with a compound of Formula (3a)

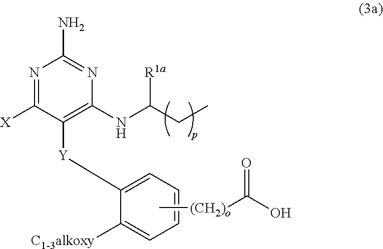

wherein

R^{1a} is H or C₁-C₄ alkyl, wherein the alkyl is optionally substituted with one to three substituents selected from the group consisting of —OZ, —NHZ, —NHAc, —COOZ, —SO₂CH₃, —SCH₃, —OCH₃,

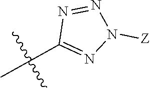

### and A¹;

X is H or C₁-C₄ alkyl, wherein the alkyl is optionally substituted with one to three substituents selected from the group consisting of A², —OZ, and —C(CH₃)₂OZ;

Y is selected from the group consisting of a bond, —CH₂—, —CF₂—,

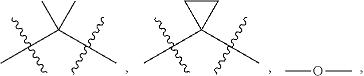

—S—, —SO₂—, —NH—, and —CH₂CH₂—;

A^{l} is selected from the group consisting of

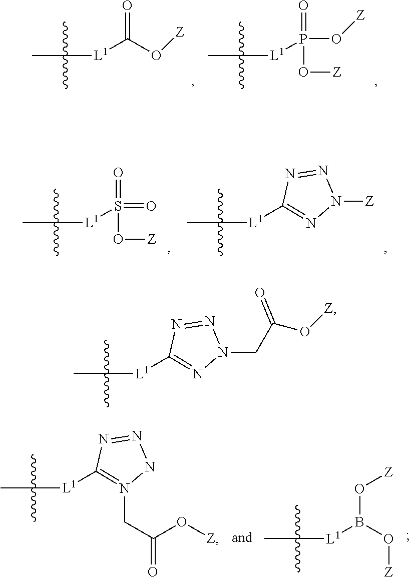

L¹ is selected from the group consisting of a bond, —(CH₂)ₙ—, —C(O)NH(CH₂)ₙ—,

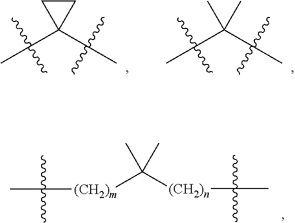

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂-[O(CH₂CH₂)]ₘ—, and —C(O)NHCH₂CH₂—[O(CH₂CH₂)]ₘ—OCH₂CH₂CF₂—;

A² is selected from the group consisting of

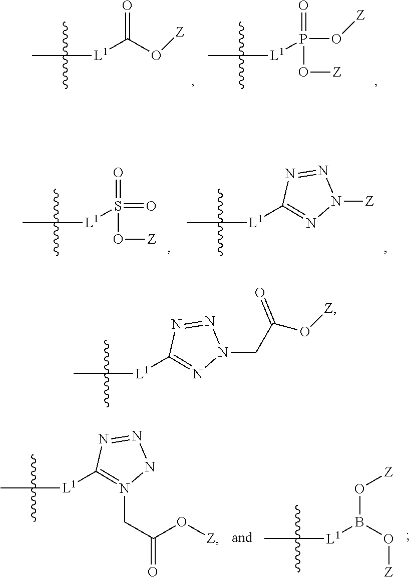

L² is selected from the group consisting of a bond, —(CH₂)ₙ—, —C(O)NH(CH₂)ₙ—,

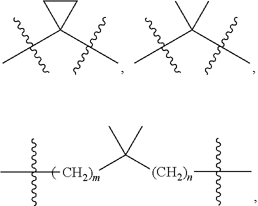

—[P(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

each Z is independently H or a protecting group;

m is an integer from zero to four; and

n and p are independently an integer from one to four; and

o is an integer from zero to four.

In some specific embodiments, Formula of 3a has the structure of Formula 3b,

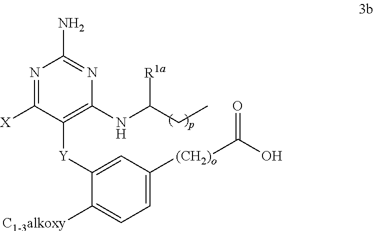

wherein R^{1a}, X, Y, p, and o are as defined for Formula 3a.

In some embodiments, the disclosure provides a method of making a peptide conjugate as described herein, or a pharmaceutically acceptable salt thereof, comprising the step of conjugating a peptide with a compound of Formula 3 or Formula 3b, wherein R^{1a} is H. In alternative embodiments, R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

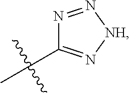 [SMILES: *c1nn[nH]n1]

and A¹. In some embodiments, R^{1a} is C₁-C₄ alkyl, wherein the alkyl is optionally substituted with —SO₂CH₃. In some embodiments, R^{1a} is C₁-C₄ alkyl, wherein the alkyl is substituted with —SO₂CH₃. In more specific embodiments, R^{1a} is

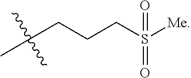 [SMILES: *CCCS(C)(=O)=O]

In some embodiments, the disclosure provides a method of making a peptide conjugate as described herein, or a pharmaceutically acceptable salt thereof, comprising the step of conjugating a peptide with a compound of Formula 3 or Formula 3b, wherein X is H. In alternative embodiments, X is C₁-C₄ alkyl. In a specific embodiment, X is methyl. In some embodiments, X is C₁-C₄ alkyl, wherein the alkyl is optionally substituted with one to three substituents selected from the group consisting of A², —OH, and —C(CH₃)₂OH.

In some embodiments, the disclosure provides a method of making a peptide conjugate as described herein, or a pharmaceutically acceptable salt thereof, comprising the step of conjugating a peptide with a compound of Formula 3 or Formula 3b, wherein Y is —CH₂—.

In some embodiments, the disclosure provides a method of making a peptide conjugate as described herein, or a pharmaceutically acceptable salt thereof, comprising the step of conjugating a peptide with a compound of Formula 3 or Formula 3b, wherein o is one. In some embodiments, o is zero. In some embodiments, o is two. In some embodiments, o is three.

In some embodiments, the disclosure provides a method of making a peptide conjugate as described herein, or a pharmaceutically acceptable salt thereof, comprising the step of conjugating a peptide with a compound of Formula 3 or Formula 3b, wherein p is one. In some embodiments, p is two. In some embodiments, p is three.

In some embodiments, the disclosure provides a method of making a peptide conjugate as described herein, or a pharmaceutically acceptable salt thereof, comprising the step of conjugating a peptide with a compound of Formula 3 or Formula 3b, wherein R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

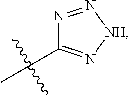 [SMILES: *c1nn[nH]n1]

### and A^{l};

X is C₁-C₄ alkyl; and

Y is —CH₂—.

In some embodiments, the disclosure provides a method of making a peptide conjugate as described herein, or a pharmaceutically acceptable salt thereof, comprising the step of conjugating a peptide with a compound of Formula 3 or Formula 3b, wherein the compound of Formula 3 has one, two, or three or more of the following features:

a) R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

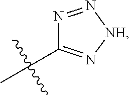 [SMILES: *c1nn[nH]n1]

### and A¹;

b) X is C₁-C₄ alkyl;

c) Y is —CH₂—;

d) C₁₋₃ alkoxy is CH₃O—;

e) o is one; and

f) p is three.

In some embodiments, the disclosure provides a method of making a peptide conjugate as described herein, or a pharmaceutically acceptable salt thereof, comprising the step of conjugating a peptide with a compound of Formula 3 or Formula 3b, wherein the compound of Formula 3 has one, two, or three or more of the following features:

a) R^{1a} is C₁-C₄ alkyl, wherein the alkyl is substituted with —SO₂CH₃;

b) X is C₁-C₄ alkyl;

c) Y is —CH₂—;

d) C₁₋₃ alkoxy is CH₃O—;

e) o is one; and

f) p is three.

In some embodiments, the disclosure provides a method of making a peptide conjugate as described herein, or a pharmaceutically acceptable salt thereof, comprising the step of conjugating a peptide with a compound of Formula 3 or Formula 3b, wherein the compound of Formula 3 has one, two, or three or more of the following features:

a) R^{1a} is

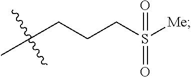 [SMILES: *CCCS(C)(=O)=O]

b) X is methyl;

c) Y is —CH₂—;

d) C₁₋₃ alkoxy is CH₃O—;

e) o is one; and

f) p is three.

It is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis,” Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of peptide conjugates and compounds disclosed herein.

In some embodiments, Z is an amino protecting group. Non-limiting examples of amino protecting groups include 9-fluorenylmethyl carbamate (Fmoc), t-butyl carbamate (BOC), benzyl carbamate (Cbz), acetamide (Ac), trifluoroacetamide, phthalamide, benzylamine (Bn), triphenylmethylamine (tritylamine, Tr), benzylideneamine, p-toluenesulfonamide (Ts, tosylamide).

In one embodiment, the disclosure provides a method of making a peptide conjugate as described herein, or a pharmaceutically acceptable salt thereof, wherein the peptide is bound to a solid phase. In another embodiment, the method further comprises the step of cleaving the conjugate from the solid phase.

The step of conjugating comprises forming a covalent bond between a peptide and a TLR7 agonist. In one embodiment, the TLR7 agonist is a low molecular weight molecule and otherwise known as a “small molecule” as opposed to a polymeric species. In one embodiment, the covalent bond is formed between the N-terminus of the peptide and a —COOH group of the TLR7 agonist. In another embodiment, the covalent bond is formed between a side chain of the peptide and the COOH group of the TLR7 agonist. In a more specific embodiment, the side chain is an amino group. In another embodiment, the side chain is a lysine residue.

Pharmaceutical Compositions

In another aspect, the disclosure provides a pharmaceutical composition comprising the peptide conjugate as described herein, or a pharmaceutically acceptable salt thereof. In some embodiments, the pharmaceutical compositions disclosed herein are tumor vaccines. The tumor vaccine may treat an existing tumor or prevent the development of a tumor.

The peptide conjugates of Formula I, Formula 1a, Formula 2a, Formula 1b, or Formula 2b, or pharmaceutically acceptable salts thereof, may be used on their own but will generally be administered in the form of a pharmaceutical composition in which the peptide conjugate of Formula I, Formula 1a, Formula 2a, Formula 1b, or Formula 2b, or a pharmaceutically acceptable salt thereof, (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

The present disclosure provides pharmaceutical compositions comprising a peptide conjugate of the present disclosure, or a pharmaceutically acceptable salt thereof, and a pharmaceutically acceptable carrier. In one embodiment, the pharmaceutically acceptable carrier is carboxy methylcellulose, saline, water, or another aqueous solution. In another embodiment, the pharmaceutically acceptable carrier is 0.1%-5% carboxy methylcellulose in water.

Depending on the mode of administration, the pharmaceutical composition will comprise from about 0.05 wt % to about 99 wt % (per cent by weight, or w/w %), more particularly from about 0.05 wt % to about 80 wt %, still more particularly from about 0.10 wt % to about 70 wt %, and even more particularly from about 0.10 wt % to about 50 wt %, of active ingredient, all percentages by weight being based on total composition.

The present disclosure also provides a pharmaceutical composition comprising a peptide conjugate of Formula I, Formula 1a, Formula 2a, Formula 1b, or Formula 2b, or a pharmaceutically acceptable salt thereof, as hereinbefore defined, in association with a pharmaceutically acceptable adjuvant, diluent or carrier.

The present disclosure further provides a process for the preparation of a pharmaceutical composition of the present disclosure which comprises mixing a peptide conjugate of Formula I, Formula 1a, Formula 2a, Formula 1b, or Formula 2b, or a pharmaceutically acceptable salt thereof, as hereinbefore defined, with a pharmaceutically acceptable adjuvant, diluent or carrier.

Methods of Use

The immune response to certain antigens can be enhanced through the use of immune potentiators, known as vaccine adjuvants. A discussion of immunological adjuvants can be found in “Current Status of Immunological Adjuvants”, Ann. Rev. Immunol., 1986, 4, pp. 369-388 and “Recent Advances in Vaccine Adjuvants and Delivery Systems” by D. T. O'Hagan and N. M. Valiante, Nat. Rev. Drug Discovery, 2003, 2(9), 727-35. The disclosures of U.S. Pat. Nos. 4,806,352; 5,026,543; and 5,026,546 describe various vaccine adjuvants appearing in the patent literature. Each of these references is hereby incorporated by reference in their entireties.

The present disclosure provides methods of administering a vaccine by administering a peptide conjugate disclosed herein alone or in combination with other agents. In another embodiment, administration of a peptide conjugate disclosed herein containing antigenic epitopes from sources such as synthetic peptides, bacterial, or viral antigens result in an immune response. In other embodiments, the present disclosure provides immunogenic compositions comprising a peptide conjugate disclosed herein effective to stimulate a cell mediated response to said one or more antigens.

In another aspect, the use of peptide conjugates disclosed herein results in an antigen dose-sparing effect. In an antigen dose-sparing effect, the same or similar efficacy of an antigen is obtained at a lower antigen dose. It also means that the effective dose of the antigen is lowered. This is a factor for antigens that have poor solubility. Antigens that are limited by poor solubility may require an antigen dose that is higher than if the antigen had good solubility and was more bioavailable. Conjugating the poorly soluble antigen to arrive at a peptide conjugate as disclosed in Formula I, Formula 1a, Formula 2a, Formula 1b, or Formula 2b, or a pharmaceutically acceptable salt thereof, may result in the same or similar efficacy of the antigen at a lower dose. Formulations of pooled neoantigen vaccines, for example, which comprise 20 or more peptides are limited by the poor solubility of the peptide. A peptide conjugate of Formula I, Formula 1a, Formula 2a, Formula 1b, or Formula 2b, or a pharmaceutically acceptable salt thereof, wherein the peptide portion comprises a pooled neoantigen vaccine may achieve the same or similar efficacy of the non-conjugated pooled neoantigen vaccine administered at a higher dose. In one embodiment, the use of a peptide conjugate disclosed herein, wherein the peptide is an antigen, lowers the effective dose of the antigen compared to the non-conjugated antigen. In another embodiment, the use of a peptide conjugate disclosed herein, wherein the peptide is an antigen, increases the immunogenicity of the antigen compared to the non-conjugated antigen.

Further provided herein is a peptide conjugate of Formula I, Formula 1a, Formula 2a, Formula 1b, or Formula 2b, or a pharmaceutically acceptable salt thereof, or a pharmaceutical composition comprising any of the foregoing peptide conjugates, or a pharmaceutically acceptable salt thereof, for use in a method of treating a disorder in a subject in need thereof. In some embodiments of the methods and uses provided herein, the disorder is a tumor.

In one aspect, the disclosure provides a method of treating a tumor in a subject in need thereof, comprising administering to the subject in need thereof a peptide conjugate described herein, or a pharmaceutically acceptable salt thereof.

In another aspect, the disclosure provides a method of vaccinating a subject in need thereof against a tumor, comprising administering to the subject in need thereof a peptide conjugate described herein, or a pharmaceutically acceptable salt thereof.

A solid tumor as used herein refers to an abnormal mass of tissue that usually does not contain cysts or liquid areas. Solid tumors may be benign or malignant. Solid tumors can occur in several places, for instance, bones, muscles, and organs. Examples of solid tumors are sarcomas, carcinomas, and lymphomas. Sarcomas are tumors in a blood vessel, bone, fat tissue, ligament, lymph vessel, muscle or tendon. Sarcomas include Ewing sarcoma and osteosarcoma, both are bone cancer sarcomas. Rhabdomyosarcoma is a soft tissue sarcoma found in muscles. Carcinomas are tumors that form in epithelial cells. Epithelial cells are found in the skin, glands and the linings of organs. Those organs includes the bladder, ureters and part of the kidneys. One common carcinoma is adrenocortical carcinoma. Common pediatric solid tumor cancers include brain tumors, neuroblastoma, Wilms tumor, rhabdomyosarcoma, retinoblastoma, osteosarcoma, and Ewing sarcoma.

In some embodiments, the tumor is a solid tumor. In some embodiments, the solid tumor is selected from the group consisting of adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, and Wilms tumor.

In some embodiments, the tumor is not a solid tumor.

In another aspect, the disclosure provides a peptide conjugate as described herein, or a pharmaceutically acceptable salt thereof, for use in therapy.

In another aspect, the disclosure provides a pharmaceutical composition as described herein, for use in therapy.

In another aspect, the disclosure provides for the use of a peptide conjugate as described herein, or a pharmaceutically acceptable salt thereof, in the manufacture of a medicament.

### EXEMPLARY EMBODIMENTS

Embodiment I-1. A peptide conjugate having the structure of Formula I, or a pharmaceutically acceptable salt thereof.

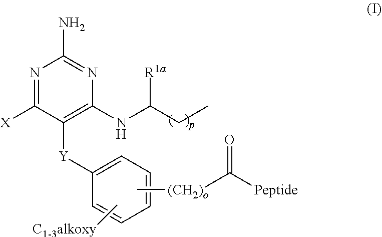

wherein

Peptide is a peptide, wherein (C═O) is attached to (i) the N-terminus of the peptide, or (ii) a side chain of the peptide where the functional group of the side chain to which C═O is attached is NH₂;

R^{1a} is H or C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

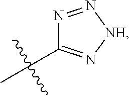 [SMILES: *c1nn[nH]n1]

### and A^{l};

X is H or C₁-C₄ alkyl, wherein the alkyl is optionally substituted with one to three substituents selected from the group consisting of A², —OH, and —C(CH₃)₂OH;

Y is selected from the group consisting of a bond, —CH₂—, —CF₂—,

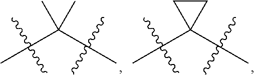

—O—, —S—, —SO₂—, —NH—, and —CH₂CH₂—;

A^{l} is selected from the group consisting of

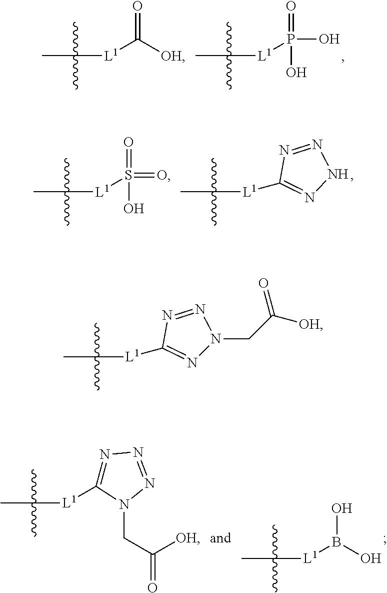

L¹ is selected from the group consisting of a bond, —(CH₂)ₙ—, —C(O)NH(CH₂)ₙ—,

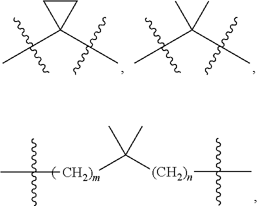

[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

A² is selected from the group consisting of

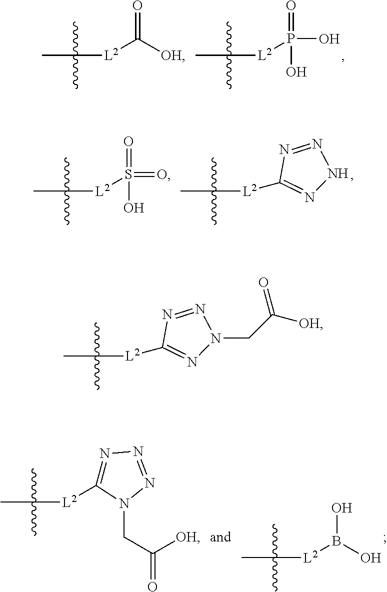

L² is selected from the group consisting of a bond, —(CH₂)ₙ—, —C(O)NH(CH₂)ₙ—,

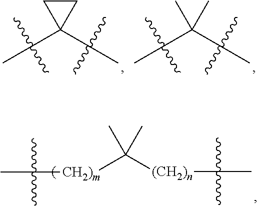

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

m is an integer from zero to four; and

n, o, and p are independently an integer from one to four.

Embodiment I-2. The peptide conjugate of Embodiment I-1, or a pharmaceutically acceptable salt thereof, having the structure of Formula (1a) or (2a), or a pharmaceutically acceptable salt thereof,

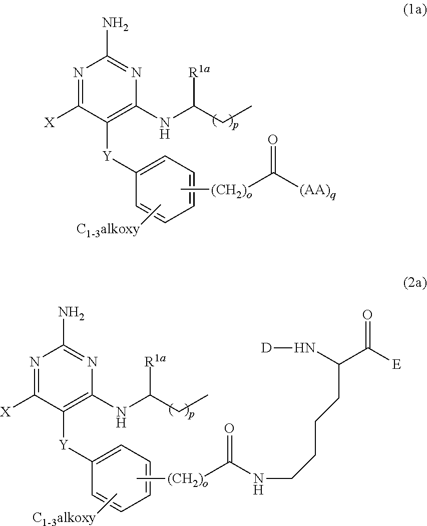

wherein

each AA is independently an amino acid, wherein the (AA)_{q} is a peptide, wherein (C═O) is attached to the N-terminus of the peptide;

q is an integer from eight to forty;

D is H or an amino acid or a peptide comprising 2 to 40 amino acids; and

E is OH or an amino acid or a peptide comprising 2 to 40 amino acids.

Embodiment I-3. The peptide conjugate of Embodiment I-2, or a pharmaceutically acceptable salt thereof, having the structure of Formula (1b) or (2b), or a pharmaceutically acceptable salt thereof,

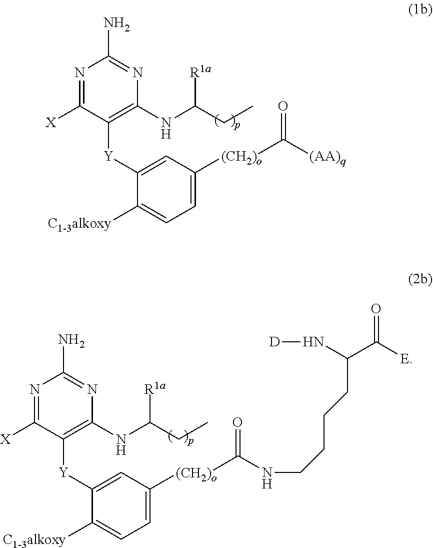

Embodiment I-4. The peptide conjugate of any one of Embodiments I-1 to I-3, or a pharmaceutically acceptable salt thereof, wherein

R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃,

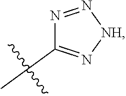 [SMILES: *c1nn[nH]n1]

### and A¹;

X is C₁-C₄ alkyl; and

Y is —CH₂—.

Embodiment I-5. The peptide conjugate of any one of Embodiments I-1 to I-4, or a pharmaceutically acceptable salt thereof, wherein R^{1a} is C₁-C₄ alkyl, and alkyl is substituted with —SO₂CH₃.

Embodiment I-6. The peptide conjugate of any one of Embodiments I-1 to I-5, or a pharmaceutically acceptable salt thereof, wherein R^{1a} is

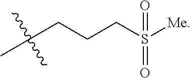 [SMILES: *CCCS(C)(=O)=O]

Embodiment I-7. The peptide conjugate of any one of Embodiments I-1 to I-6, or a pharmaceutically acceptable salt thereof, wherein X is methyl.

Embodiment I-8. The peptide conjugate of any one of Embodiments I-1 to I-7, or a pharmaceutically acceptable salt thereof, wherein p is three.

Embodiment I-9. The peptide conjugate of any one of Embodiments I-1 to I-8, or a pharmaceutically acceptable salt thereof, wherein o is one.

Embodiment I-10. The peptide conjugate of any one of Embodiments I-1 to I-9, or a pharmaceutically acceptable salt thereof, wherein Y is —CH₂—.

Embodiment I-11. The peptide conjugate of any one of Embodiments I-1 to I-10, or a pharmaceutically acceptable salt thereof, wherein the peptide is an antigen.

Embodiment I-12. The peptide conjugate of Embodiment I-11, or a pharmaceutically acceptable salt thereof, wherein the antigen is a bacterial or viral antigen.

Embodiment I-13. The peptide conjugate of Embodiment I-11, or a pharmaceutically acceptable salt thereof, wherein the antigen is an epitope.

Embodiment I-14. The peptide conjugate of Embodiment I-11, or a pharmaceutically acceptable salt thereof, wherein the antigen is a shared tumor antigen.

Embodiment I-15. The peptide conjugate of Embodiment I-11, or a pharmaceutically acceptable salt thereof, wherein the antigen is a personalized neoantigen.

Embodiment I-16. The peptide conjugate of any one of Embodiments I-1 to I-10, or a pharmaceutically acceptable salt thereof, wherein the peptide is a vaccine.

Embodiment I-17. A peptide conjugate of any one of Embodiments I-1 to I-16, or a pharmaceutically acceptable salt thereof, wherein the peptide is prepared by solid phase synthesis.

Embodiment I-18. A pharmaceutical composition comprising the peptide conjugate of any one of Embodiments I-1 to I-17, or a pharmaceutically acceptable salt thereof

Embodiment I-19. A method of making a conjugate of any one of Embodiment I-1 to I-16, or a pharmaceutically acceptable salt thereof, comprising the step of conjugating a peptide with a compound of Formula (3a)

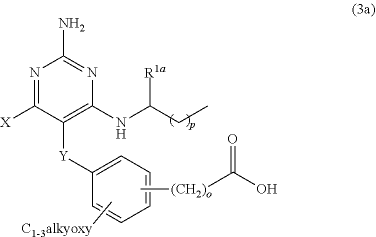

wherein

R^{1a} is H or C₁-C₄ alkyl, wherein the alkyl is optionally substituted with one to three substituents selected from the group consisting of —OZ, —NHZ, —NHAc, —COOZ, —SO₂CH₃, —SCH₃, —OCH₃,

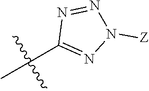

### and A¹;

X is H or C₁-C₄ alkyl, wherein the alkyl is optionally substituted with one to three substituents selected from the group consisting of A², —OZ, and —C(CH₃)₂OZ;

Y is selected from the group consisting of a bond, —CH₂—, —CF₂—,

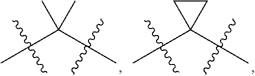

—O—, —S—, —SO₂—, —NH—, and —CH₂CH₂—;

A¹ is selected from the group consisting of

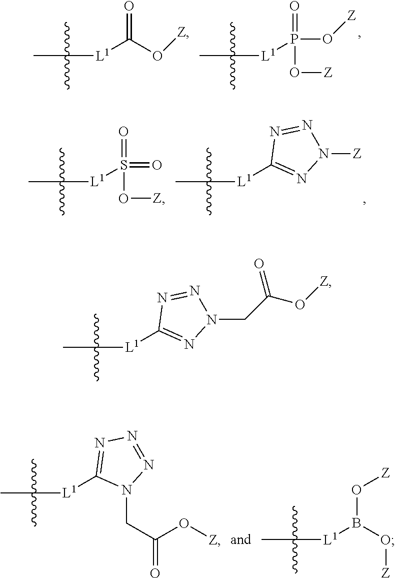

L¹ is selected from the group consisting of a bond, —(CH₂)ₙ—, —C(O)NH(CH₂)ₙ—,

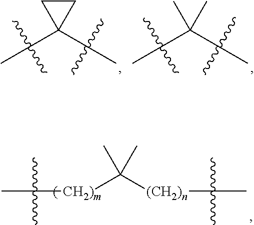

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

A² is selected from the group consisting of

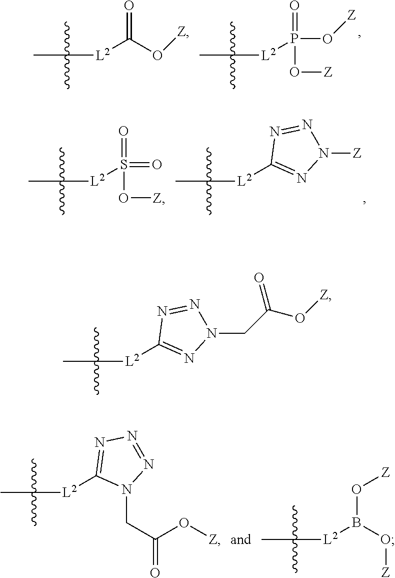

L² is selected from the group consisting of a bond, —(CH₂)ₙ—, —C(O)NH(CH₂)ₙ—,

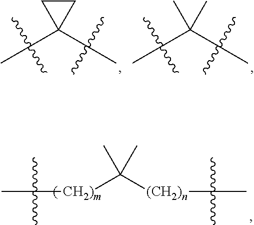

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

each Z is independently H or a protecting group;

m is an integer from zero to four; and

n, o, and p are independently an integer from one to four.

Embodiment I-20. The method according to Embodiment I-19, wherein the peptide is bound to a solid phase.

Embodiment I-21. The method according to Embodiment I-20, further comprising the step of cleaving the conjugate from the solid phase.

Embodiment I-22. A peptide conjugate of any one of Embodiments I-1 to I-17, or a pharmaceutically acceptable salt thereof, for use in therapy.

Embodiment I-23. A pharmaceutical composition of Embodiment I-18, for use in therapy.

Embodiment I-24. Use of a peptide conjugate of any one of Embodiments I-1 to I-17, or a pharmaceutically acceptable salt thereof, in the manufacture of a medicament.

Embodiment II-1. A peptide conjugate having the structure of Formula I, or a pharmaceutically acceptable salt thereof,

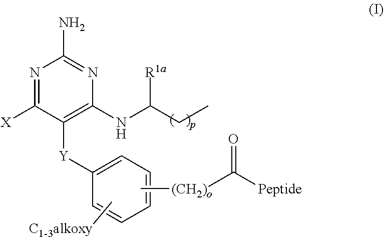

wherein

Peptide is a peptide, wherein (C═O) is attached to (i) the N-terminus of the peptide, or (ii) a side chain of the peptide where the functional group of the side chain to which C═O is attached is NH₂;

R^{1a} is H or C₁-C₄ alkyl, wherein the alkyl is optionally substituted with one to three substituents selected from the group consisting of —OH, —NH₂, —NHAc, —COOH, —SO₂CH₃,

—SCH₃, —OCH₃,

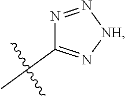 [SMILES: *c1nn[nH]n1]

### and A¹;

X is H or C₁-C₄ alkyl, wherein the alkyl is optionally substituted with one to three substituents selected from the group consisting of A², —OH, and —C(CH₃)₂OH;

Y is selected from the group consisting of a bond, —CH₂—, —CF₂—,

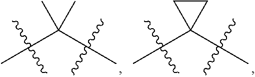

—O—, —S—, —SO₂—, —NH—, and —CH₂CH₂—;
A¹ is selected from the group consisting of

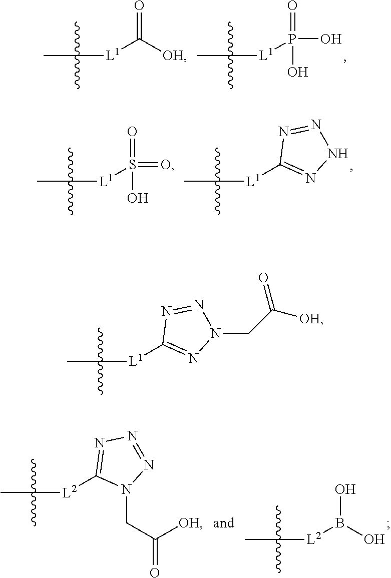

L¹ is selected from the group consisting of a bond, —(CH₂)ₙ—, —C(O)NH(CH₂)ₙ—,

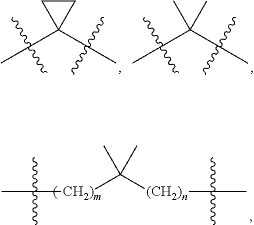

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

A² is selected from the group consisting of

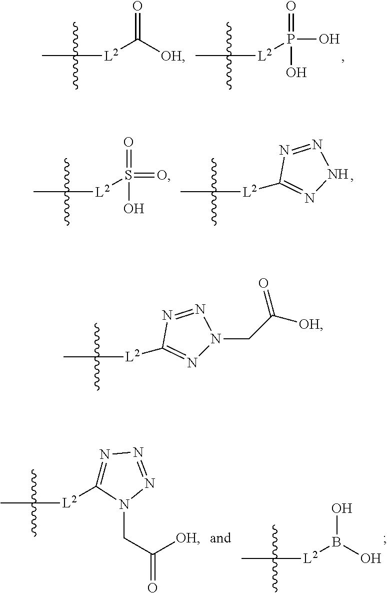

L² is selected from the group consisting of a bond, —(CH₂)ₙ—, —C(O)NH(CH₂)ₙ—,

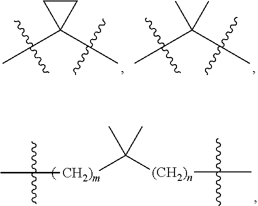

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O NHCH₂CH₂—[O(CH₂CH₂)]ₘ—OCH₂CH₂CF₂—;

m is an integer from zero to four;

n and p are independently an integer from one to four; and

o is an integer from zero to four.

Embodiment II-2. The peptide conjugate of Embodiment II-1, or a pharmaceutically acceptable salt thereof, having the structure of Formula (1a) or (2a), or a pharmaceutically acceptable salt thereof,

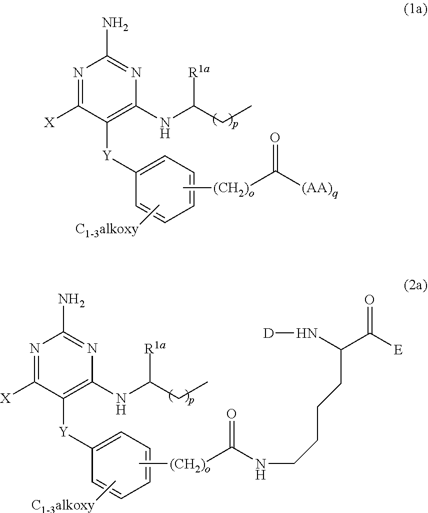

wherein
each AA is independently an amino acid, wherein the (AA)_{q} is a peptide, wherein (C═O) is attached to the N-terminus of the peptide;
q is an integer from eight to forty;
D is H or an amino acid or a peptide comprising 2 to 40 amino acids; and
E is OH or an amino acid or a peptide comprising 2 to 40 amino acids.

Embodiment II-3. The peptide conjugate of Embodiment II-2, or a pharmaceutically acceptable salt thereof, having the structure of Formula (1b) or (2b), or a pharmaceutically acceptable salt thereof,

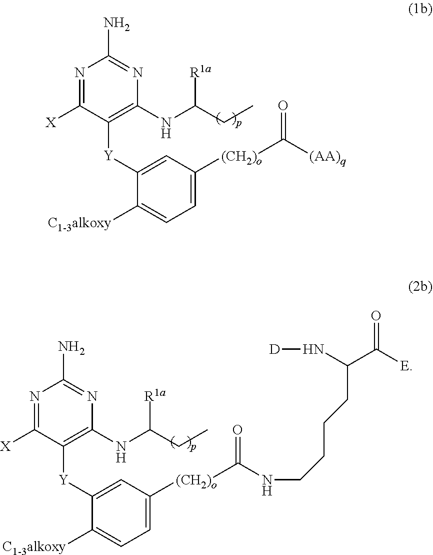

Embodiment II-4. The peptide conjugate of any one of Embodiments II-1 to II-3, or a pharmaceutically acceptable salt thereof, wherein

R^{1a} C₁-C₄ alkyl, wherein the alkyl is optionally substituted with one to three substituents selected from the group consisting of —OH, —NH₂, —NHAc, —COOH, —SO₂CH₃, —SCH₃, —CH₃,

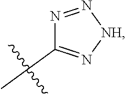 [SMILES: *c1nn[nH]n1]

### and A¹;

X is C₁-C₄ alkyl; and

### Y is —CH₂—.

Embodiment II-5. The peptide conjugate of any one of Embodiments II-1 to II-4, or a pharmaceutically acceptable salt thereof, wherein R^{1a} is C₁-C₄ alkyl, and alkyl is substituted with —SO₂CH₃.

Embodiment II-6. The peptide conjugate of any one of Embodiments II-1 to II-5, or a pharmaceutically acceptable salt thereof, wherein R^{1a} is

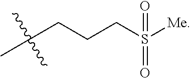 [SMILES: *CCCS(C)(=O)=O]

Embodiment II-7. The peptide conjugate of any one of Embodiments II-1 to II-6, or a pharmaceutically acceptable salt thereof, wherein X is methyl.

Embodiment II-8. The peptide conjugate of any one of Embodiments II-1 to II-7, or a pharmaceutically acceptable salt thereof, wherein p is three.

Embodiment II-9. The peptide conjugate of any one of Embodiments II-1 to II-8, or a pharmaceutically acceptable salt thereof, wherein o is one.

Embodiment II-10. The peptide conjugate of any one of Embodiments II-1 to II-9, or a pharmaceutically acceptable salt thereof, wherein Y is —CH₂—.

Embodiment II-11. The peptide conjugate of any one of Embodiments II-1 to II-10, or a pharmaceutically acceptable salt thereof, wherein the peptide is an antigen.

Embodiment II-12. The peptide conjugate of Embodiment II-11, or a pharmaceutically acceptable salt thereof, wherein the antigen is a bacterial or viral antigen.

Embodiment II-13. The peptide conjugate of Embodiment II-11, or a pharmaceutically acceptable salt thereof, wherein the antigen is an epitope.

Embodiment I-14. The peptide conjugate of Embodiment II-11, or a pharmaceutically acceptable salt thereof, wherein the antigen is a shared tumor antigen.

Embodiment II-15. The peptide conjugate of Embodiment II-11, or a pharmaceutically acceptable salt thereof, wherein the antigen is a personalized neoantigen.

Embodiment II-16. The peptide conjugate of any one of Embodiments II-1 to II-10, or a pharmaceutically acceptable salt thereof, wherein the peptide is a vaccine.

Embodiment II-17. A peptide conjugate of any one of Embodiments II-1 to II-16, or a pharmaceutically acceptable salt thereof, wherein the peptide is prepared by solid phase synthesis.

Embodiment II-18. A pharmaceutical composition comprising the peptide conjugate of any one of Embodiments II-1 to II-17, or a pharmaceutically acceptable salt thereof

Embodiment II-19. A method of treating a tumor in a subject in need thereof, comprising administering to the subject in need thereof a peptide conjugate, or a pharmaceutically acceptable salt thereof, of any one of Embodiments II-1 to II-17, or a pharmaceutical composition of Embodiment II-18.

Embodiment II-20. A method of vaccinating a subject in need thereof against a tumor, comprising administering to the subject in need thereof a peptide conjugate, or a pharmaceutically acceptable salt thereof, of any one of Embodiments II-1 to II-17, or a pharmaceutical composition of Embodiment II-18.

Embodiment II-21. The method of Embodiment II-19 or 20, wherein the tumor is a solid tumor.

Embodiment II-22. A method of making a conjugate of any one of Embodiments II-1 to II-17, or a pharmaceutically acceptable salt thereof, comprising the step of conjugating a peptide with a compound of Formula (3a)

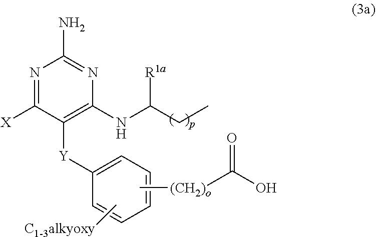

wherein

wherein R^{1a} is H or C₁-C₄ alkyl, wherein the alkyl is optionally substituted with one to three substituents selected from the group consisting of —OZ, —NHZ, —NHAc, —COOZ, —SO₂CH₃, —SCH₃, —OCH₃,

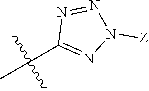

### and A^{l};

X is H or C₁-C₄ alkyl, wherein the alkyl is optionally substituted with one to three substituents selected from the group consisting of A², —OZ, and —C(CH₃)₂OZ;

Y is selected from the group consisting of a bond, —CH₂—, —CF₂—,

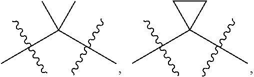

—O—, —S—, —SO₂—, —NH—, and —CH₂CH₂—;

A¹ is selected from the group consisting of

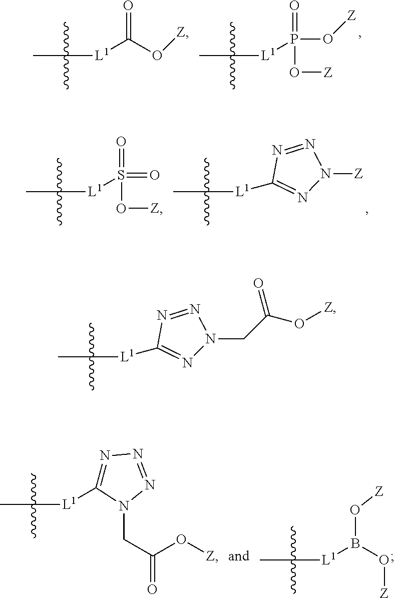

L¹ is selected from the group consisting of a bond, —(CH₂)ₙ—, —C(O)NH(CH₂)ₙ—,

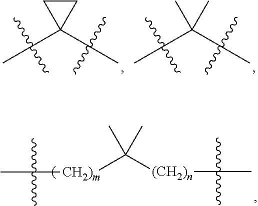

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;

A² is selected from the group consisting of

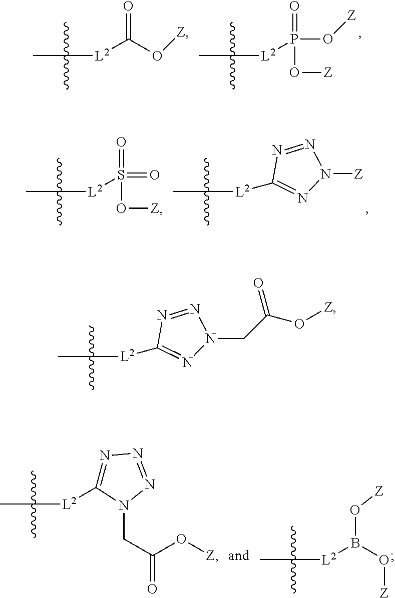

L² is selected from the group consisting of a bond, —(CH₂)ₙ—, —C(O)NH(CH₂)ₙ—,

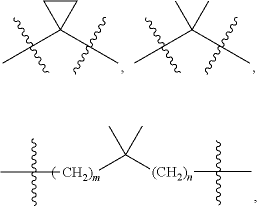

—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH2CH₂)]ₘ—, and —C(O)NHCH₂CH₂—[O(CH₂CH₂)]ₘ—OCH₂CH₂CF₂—;

each Z is independently H or a protecting group;

m is an integer from zero to four;

n and p are independently an integer from one to four; and

o is an integer from zero to four.

Embodiment II-23. The method according to Embodiment I-22, wherein the peptide is bound to a solid phase.

Embodiment II-24. The method according to Embodiment I-23, further comprising the step of cleaving the conjugate from the solid phase.

Embodiment II-25. A peptide conjugate of any one of Embodiments II-1 to II-17, or a pharmaceutically acceptable salt thereof, for use in therapy.

Embodiment II-26. A pharmaceutical composition of Embodiment I-18, for use in therapy.

Embodiment II-27. Use of a peptide conjugate of any one of Embodiments II-1 to II-17, or a pharmaceutically acceptable salt thereof, in the manufacture of a medicament.

### EXAMPLES

The following examples are provided to illustrate the present disclosure, and should not be construed as limiting thereof. In these examples, all parts and percentages are by weight, unless otherwise noted. Abbreviations in the examples are noted below.

### Abbreviations

| | |
|---|---|
| AIBN | azobisisobutyronitrile |
| aq. | aqueous |
| DCM | dichloromethane |
| DMAP | 4-dimethylaminopyridine |
| EA | ethyl acetate |
| Eq | equivalent |
| h or hr | hour |
| HPLC | high performance liquid chromatography |
| LC-MS | liquid chromatography mass spectrometry |
| min | minutes |
| NBS | N-bromosuccinimide |
| NMP | N-methylpyrrolidine |
| NMR | nuclear magnetic resonance |
| PE | petroleum ether |
| prep | preparative |
| rt or r.t. | room temperature |
| sat. | saturated |
| TBAF | tetrabutylammonium flouride |
| TBS | tert-butyldimethylsilyl |
| TEA | triethylamine |
| TFA | trifluoroacetic acid |
| THF | tetrahydrofuran |
| TLC | thin layer chromatography |
| HPLC | high performance liquid chromatography |
| LC-MS | liquid chromatography mass spectrometry |

### CHEMISTRY SYNTHESIS EXAMPLES

### Synthesis Example 1: Synthesis of a TLR7 Agonist, Compound 1, for use in a Peptide Conjugate

### Example 1a: Synthesis of a TLR7 Agonist, Compound 1

This example describes the preparation of Compound 1, (S)-2-(3-((2-amino-4-methyl-6-((1-(methylsulfonyl)heptan-3-yl)amino)pyrimidin-5-yl)methyl)-4- methoxyphenyl)acetic acid. Once obtained, this TLR7 agonist can be conjugated onto the alpha-amino group of any amino acids or side chain amino group of lysine.

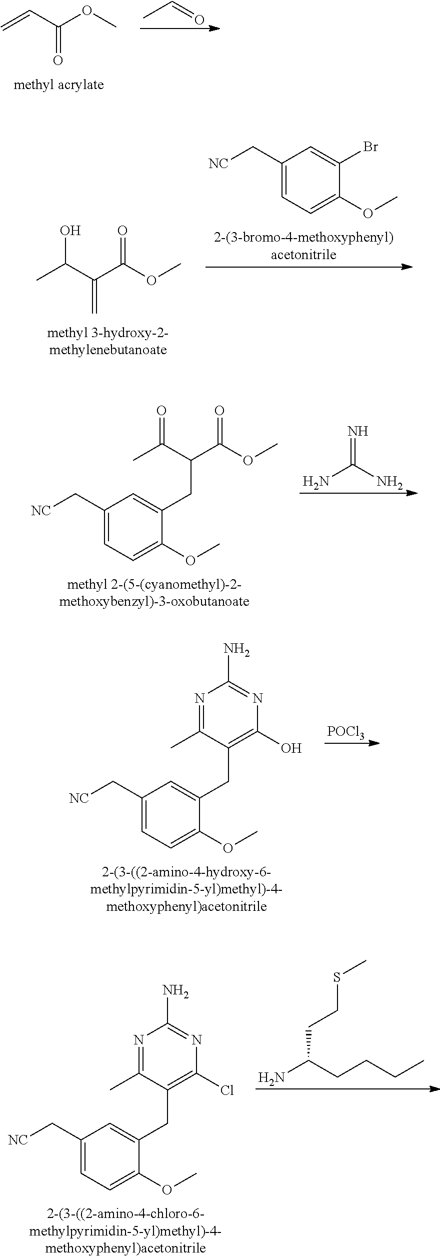

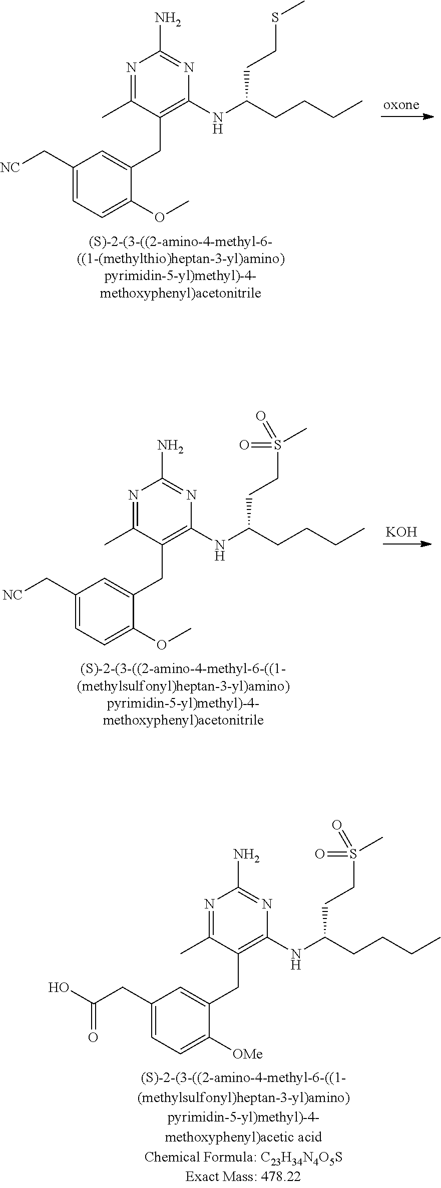

### Step 1: methyl 3-hydroxy-2-methylenebutanoate

A mixture of methyl acrylate (1.0 eq) in 1:1 dioxane/water (8.5M), acetaldehyde (3.0 eq) and DABCO (1.0 eq) was stirred overnight at r.t. The mixture was partitioned between DCM/water. The organic layer was dried over Na₂SO₄, concentrated and purified by flash chromatography on silica (eluent PE/EA=100:1˜2:1) to give the title compound.

### Step 2: methyl 2-(5-(cyanomethyl)-2-methoxybenzyl)-3-oxobutanoate

A mixture of 2-(3-bromo-4-methoxyphenyl)acetonitrile (1.0 eq) in MeCN (0.44M), methyl 3-hydroxy-2-methylenebutanoate (2.0 eq), PdCl₂(P(o-tol)₃)₂) (0.03 eq), TEA (2.0 eq) was stirred overnight at 70° C. The mixture was partitioned between EA/water. The organic layer was dried over Na₂SO₄, concentrated and purified by flash chromatography on silica (eluent PE/EA =100:1˜2:1) to give the title compound.

### Step 3: 2-(3(2-amino-4-hydroxy-6-methylpyrimidin-5-yl)methyl)-4-methoxyphenyl)acetonitrile

A mixture of methyl 2-(5-(cyanomethyl)-2-methoxybenzyl)-3-oxobutanoate (1.0 eq), guanidine carbonate (1.0 eq) in MeOH (0.46M) was stirred overnight at 65° C. After cooled down to r.t., the reaction mixture was filtered. The filter cake was dried under vacuum to give the title compound.

### Step 4: 2-(3-((2-amino-4-chloro-6-methylpyrimidin-5-yl)methyl)-4-methoxyphenyl)acetonitrile

To a solution of 2-(3-((2-amino-4-hydroxy-6-methylpyrimidin-5-yl)methyl)-4-methoxyphenyl)acetonitrile (1.0 eq) in POCl₃ (0.9M) was stirred at 100° C. for 16 h under N₂. The reaction mixture was cooled to r.t., and POCl₃ was evaporated under reduced pressure. The residue was diluted with water. pH value was adjusted to 8 by adding solid NaHCO₃. Then the mixture was stirred at 50° C. for 1 h, cooled to r.t., and the precipitate was collected by filtration. The filter cake was washed with water, dried in vacuum to give the title compound as a white solid.

### Step 5: (S)-2-(3-(2-amino-4-methyl-6-((1-(methylthio)heptan-3-yl)amino)pyrimidin-5-yl)methyl)-4-methoxyphenyl)acetonitrile

A mixture of 2-(3-((2-amino-4-chloro-6-methylpyrimidin-5-yl)methyl)-4-methoxyphenyl)acetonitrile (1.0 eq) in NMP (1.7M) and (S)-1-(methylthio)heptan-3-amine (1.5 eq) was stirred at 120° C. for 16 h under nitrogen. The mixture was diluted with water, and the aqueous phase was extracted with EA. The organic layer was washed with water and brine, dried over Na₂SO₄, and concentrated. The crude product was purified by column chromatography (DCM/MeOH=50:1) to give the title compound as a yellow solid.

### Step 6: (S)-2-(3-((2-amino-4-methyl-6-((1-(methylsulfonyl)heptan-3-yl)amino)pyrimidin-5-yl)methyl)-4-methoxyphenyl)acetonitrile

To a solution of (S)-2-(3-((2-amino-4-methyl-6-((1-(methylthio)heptan-3-yl)amino)-pyrimidin-5-yl)methyl)-4-methoxyphenyl)acetonitrile (1 eq) in 1:1:1 THF/MePH/H₂O (0.2M) was added oxone (1.2 eq) in portions at r.t. The reaction was stirred at r.t. for 2 h, and then diluted with DCM. The organic layer was washed with water and brine, dried, and concentrated to give a light yellow solid, which was used in the next step directly.

### Step 7: (S)-2-(3-((2-amino-4-methyl-6-((1-(methylsulfonyl)heptan-3-yl)amino)pyrimidin-5-yl)methyl)-4-methoxyphenyl)acetic Acid

To a solution of (S)-2-(3-((2-amino-4-methyl-6-((1-(methylsulfonyl)heptan-3-yl)amino)pyrimidin-5-yl)methyl)-4-methoxyphenyl)acetonitrile (1.0 eq) in 1:1 MeOH/H₂O (0.1M) was added KOH (7.5 eq). The reaction was stirred at 120° C. for 4 h. Solvent was removed and HCl was added to achieve pH 9. The mixture was purified by prep-HPLC (0.1%NH₃.H₂O/CH₃CN) to give the title Compound 1 as a light yellow solid. LCMS: [M+H]⁺=479.3 ¹H NMR (400 MHz, CD₃OD) δ 7.16 (dd, J=8.4, 2.0 Hz, 1H), 6.98 (d, J=2.0 Hz, 1H), 6.91 (d, J=8.4 Hz, 1H), 4.30-4.22 (m, 1H), 3.89 (s, 3H), 3.77-3.68 (m, 2H), 3.32 (s, 2H), 2.91-2.70 (m, 5H), 2.32 (s, 3H), 2.08-1.95 (m, 1H), 1.81-1.69 (m, 1H), 1.56-1.05 (m, 6H), 0.83 (t, J=7.2 Hz, 3H).

### Example 1b: Synthesis of Compound Intermediate (S)-1-(methylthio)heptan-3-amine

The scheme below describes the synthesis of the compound intermediate (S)-1-(methylthio)heptan-3-amine, which is used in the preparation of the TLR7 agonist described above (Compound 1).

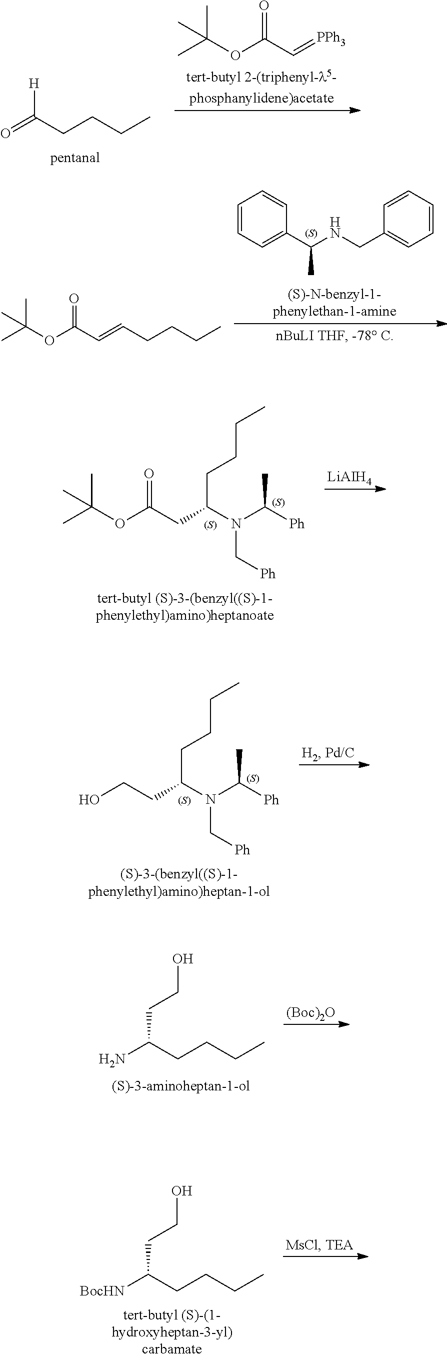

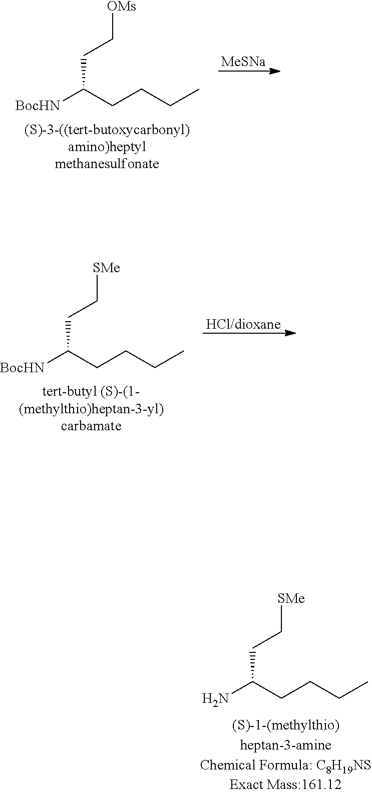

### Step 1: tert-butyl (E)-hept-2-enoate

A mixture of pentanal (1.0 eq) and tert-butyl 2-(triphenyl-λ⁵-phosphanylidene)acetate (1.05 eq) in THF (1M) was stirred at 50° C. for 16 hr. The mixture was concentrated under reduced pressure, and PE was added. The solids were filtered off, and the filtrate was evaporated to dryness. The crude residue was purified by flash column chromatography (eluent: PE to PE/EA=100:1) to give the title compound as a yellow oil.

### Step 2: tert-butyl (S)-3-(benzyl((S)-1-phenylethyl)amino)heptanoate

To a solution of (S)-N-benzyl-1-phenylethan-1-amine (1.3 eq) in THF (0.9M) at −78° C. was added dropwise 2.5M n-BuLi (1.2 eq) over 20 min. The mixture was stirred at −78° C. for 10 min, then tert-butyl (E)-hept-2-enoate (1.0 eq) in THF (0.7M) was added dropwise. The resulting mixture was stirred at −78° C. for 30 min, then quenched with aq. NH₄Cl and extracted with EA. The organic layer was dried over Na₂SO₄, filtered, and concentrated under reduced pressure. The residue was purified by flash column chromatography (eluent: PE to PE/EA=100:1) to give the title compound as a yellow oil.

### Step 3: (S)-3-(benzyl((S)-1-phenylethyl)amino)heptan-1-ol

To a solution of tert-butyl (S)-3-(benzyl((S)-1-phenylethyl)amino)heptanoate (1.0 eq) in anhydrous THF (0.4M) at 0°C. was added portion-wise LiAlH₄ (1.6 eq). After the addition, the mixture was stirred at r.t. for 5 hr. The mixture was quenched with 1.0 M NaOH solution, filtered, and the filtrate was extracted with EA. The organic layer was dried over Na₂SO₄, concentrated under reduced pressure. The residue was purified by flash column chromatography (eluent: PE to PE/EA=5:1) to give the title compound as a yellow oil.

### Step 4: (S)-3-aminoheptan-1-ol

To a solution of (S)-3-(benzyl((S)-1-phenylethyl)amino)heptan-1-ol (1.0 eq) in MeOH (0.1M) was stirred at 50° C. for 48 hr under H₂ atmosphere in presence of Pd/C (10% wt). The mixture was filtered, and the filtrate was concentrated under reduced pressure to give the title compound as a brown oil. Step 5: tert-butyl (S)-(1-hydroxyheptan-3-yl)carbamate

To a solution of (S)-3-aminoheptan-1-ol (1.0 eq) in 1:1 dioxane/H₂O (0.5M) at 0° C. was added NaOH (1.2 eq) and Boc₂O (1.2 eq) and warmed to rt. After the reaction was completed, the mixture was partitioned between H₂O/EA. The organic layer was dried over Na₂SO₄, concentrated under reduced pressure. The residue was purified by flash column chromatography (eluent: PE to PE/EA=3:1) to give the title compound as white solid.

### Step 6: (S)-3-((tert-butoxycarbonyl)amino)heptyl methanesulfonate

A mixture of tert-butyl (S)-(1-hydroxyheptan-3-yl)carbamate (1.0 eq) and TEA (1.2 eq) in DCM (0.4M) was added dropwise methanesulfonyl chloride (1.1 eq.) and stirred at 0° C. for 1 hr. The resulting mixture was partitioned between EA and water. The organic layer was dried over Na₂SO₄, concentrated under reduced pressure to give the title compound as a brown oil.

### Step 7: tert-butyl (S)-(1-(methylthio)heptan-3-yl)carbamate

A mixture of (S)-3-((tert-butoxycarbonyl)amino)heptyl methanesulfonate (1.0 eq) in DMF (0.7M) and MeSNa (2.0 eq) was stirred at 70° C. for 16 hr. The resulting mixture was partitioned between EA and water. The organic layer was dried over Na₂SO₄, concentrated under reduced pressure. The residue was purified by flash column chromatography (eluent: PE to PE/EA=10:1) to give the title compound as a yellowish oil. Step 8: (S)-1-(methylthio)heptan-3-amine

To a solution of tert-butyl (S)-(1-(methylthio)heptan-3-yl)carbamate (1.0 eq) in DCM (0.5M) was added excess 4M HCl/dioxane (⅓ volume equivalent). The resulting mixture was stirred at r.t. for 16 hr, concentrated under reduced pressure. The residue was triturated with Et₂O and the precipitated solid was collected by filtration to give the title compound as a white solid (HCl salt). LC-MS: [M+H]⁺=162 ¹H NMR (400 MHz, CDCl₃) δ 8.43 (br s, 3H), 3.41-3.38 (m, 1H), 2.71 (t, J=6.8 Hz, 2H), 2.13 (s, 3H), 2.11-2.03 (m, 1H), 2.00-1.91 (m, 1H), 1.82-1.66 (m, 2H), 1.52-1.32 (m, 4H), 0.92 (t, J=7.2 Hz, 3H).

### Synthesis Example 2: Conjugation of a TLR7 Agonist with an Amino Acid

This example demonstrates the conjugation of the TLR7 agonist Compound 1 (Synthesis Example 1) with the alpha-amino group of alanine using HATU chemistry.

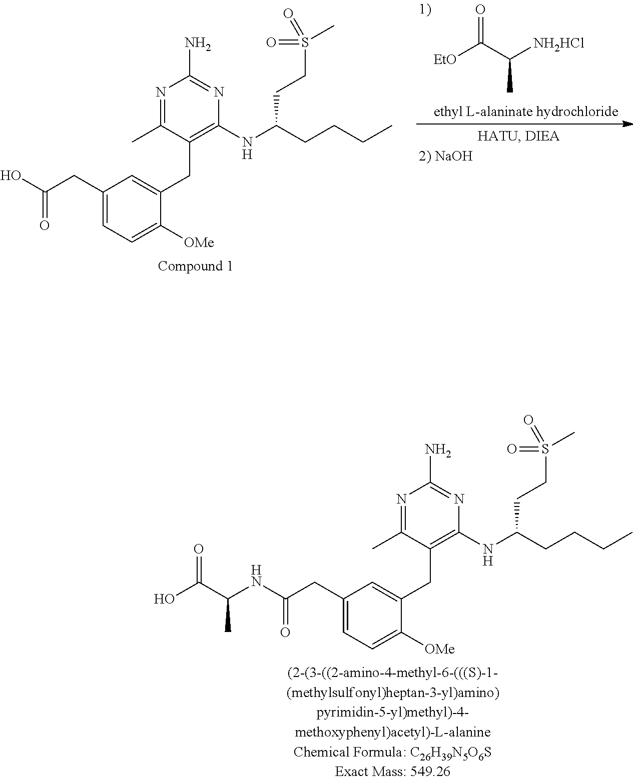

To a solution of Compound 1 (1.0 eq) in DMF (0.04M), was added ethyl L-alaninate hydrochloride (1.0 eq), Hexafluorophosphate Azabenzotriazole Tetramethyl Uronium (HATU, 2.0 eq), and N,N-Diisopropylethylamine (DIEA, 4.0 eq) at room temperature. The reaction was stirred for 4 h under nitrogen. The reaction was diluted with ethyl acetate, and the organic layer was washed with brine, dried, and concentrated. The crude was dissolved in 2:1 MeOH/H₂O (0.04M) and added excess NaOH (30.0 eq). The reaction was stirred at 80° C. for 2 h. The reaction mixture was purified by prep-HPLC (0.1%NH_{3/}CH₃CN/H₂O) to give the title compound as a white solid. LCMS: [M+1]⁺=550 ¹H NMR (400 MHz, MeOD) δ 7.18 (dd, J=8.4, 1.6 Hz, 1H), 6.98 (d, J=8.4 Hz, 1H), 6.91 (d, J =1.6 Hz, 1H), 4.45-4.35 (m, 1H), 4.17-4.11 (m, 1H), 3.92 (s, 3H), 3.85-3.74 (m, 2H), 3.41 (s, 2H), 3.01-2.80 (m, 5H), 2.32 (s, 3H), 2.09-2.01 (m, 1H), 1.92-1.83 (m, 1H), 1.58-1.44 (m, 2H), 1.32-1.05 (m, 7H), 0.83 (t, J=6.8 Hz, 3H).

### Synthesis Example 3: Conjugation of a TLR7 Agonist with an Amino Acid Side Chain

This example demonstrates the conjugation of the TLR7 agonist Compound 1 (Synthesis Example 1) with the side chain amino group of lysine using NHS ester chemistry.

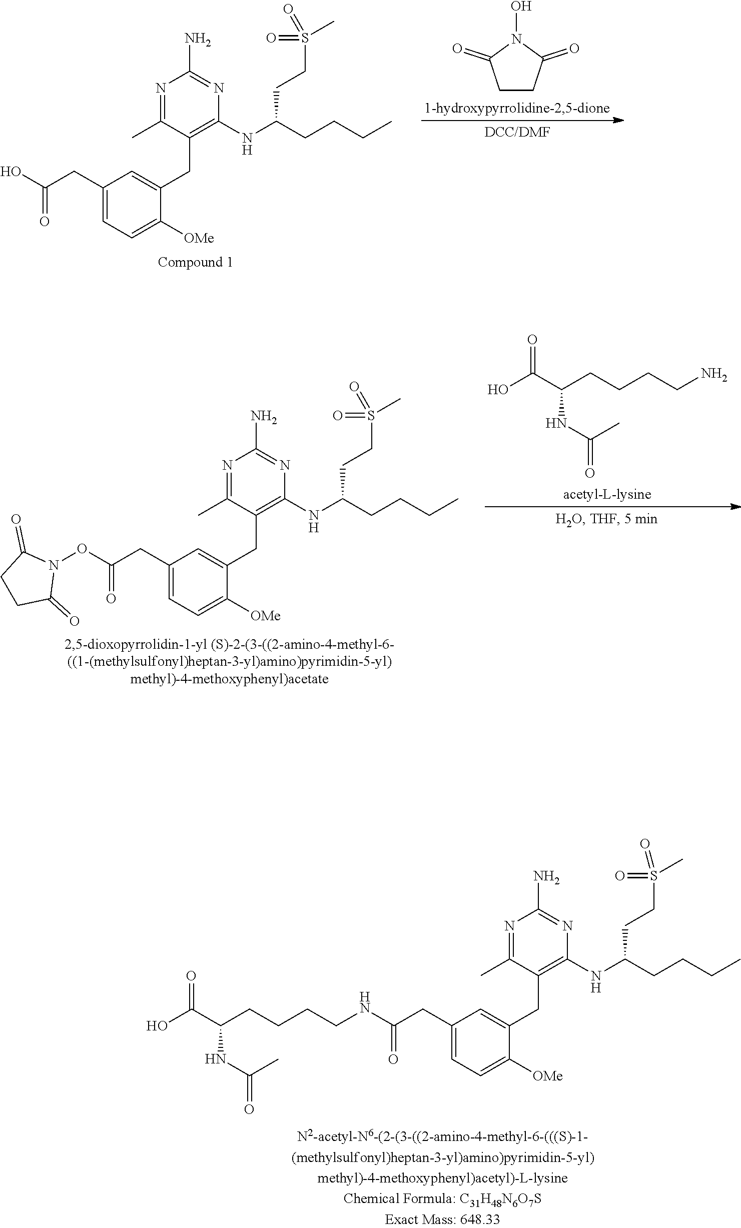 [SMILES: CCCC[C@@H](CCS(C)(=O)=O)Nc1nc(N)nc(C)c1Cc1cc(CC(=O)O)ccc1OC ; O=C1CCC(=O)N1O ; CCCC[C@@H](CCS(C)(=O)=O)Nc1nc(N)nc(C)c1Cc1cc(CC(=O)ON2C(=O)CCC2=O)ccc1OC ; CC(=O)N[C@@H](CCCCN)C(=O)O ; CCCC[C@@H](CCS(C)(=O)=O)Nc1nc(N)nc(C)c1Cc1cc(CC(=O)NCCCC[C@H](NC(C)=O)C(=O)O)ccc1OC]

To a solution of Compound 1 (1.0 eq) in DMF (0.36M) and 1-hydroxypyrrolidine-2,5-dione (2 eq) was added DCC (2.0 eq) at r.t. The reaction was stirred at r.t. for 16 h under nitrogen. The solid was filtered off. The filtrate was diluted with EA, and the organic layer was washed with brine, dried, and concentrated. The crude product was purified by column chromatography (DCM:MeOH=10:1) to give the NHS ester. The NHS ester was reacted with acetyl-L-lysine (15 eq) in 2:1 H₂O/THF at r.t. The reaction proceeded rapidly and was completed within 5 minutes. The mixture was purified by prep-HPLC (mobile phase: NH₃/CH₃CN/H₂O) to give the title compound as a white solid. LCMS: [M+1]⁺=649 ¹H NMR (400 MHz, CD₃OD) δ 7.18 (dd, J=8.4 Hz 2.0 Hz, 1H), 6.97 (d, J=8.4 Hz, 1H), 6.74 (d, J=2.0 Hz, 1H), 4.46-4.40 (m, 1H), 4.23 (t, J=6.4 Hz, 1H), 3.93 (s, 3H), 3.85-3.74 (m, 2H), 3.33 (s, 2H), 3.20-2.80 (m, 7H), 2.29 (s, 3H), 2.15-201 (m, 1H), 1.98 (s, 3H), 1.95-1.69 (m, 3H), 1.62-1.05 (m, 10H), 0.83 (t, J=7.2 Hz, 3H).

### Synthesis Example 4: Peptide Conjugation

This example describes the preparation of peptide conjugates based on mouse epitopes and the TLR7 agonist described in Synthesis Example 1 (Compound 1).

1. Peptide Sequences

The following is a list of 12 Balb/c H-2^{d} epitopes used in the peptide conjugates. The epitopes are predicted from IEDB (http://www.iedb.org)

| SEQ | Peptide | | | | Epitope |
|---|---|---|---|---|---|
| ID NO | ID | MHC | Loci | Sequence | ID |
| SEQ ID | AP1 | I | H-2Dd | HSNLNDTTYQRTR | 67436 |
| NO: 1 | | | | ALVRTGMDPRMC | |
| SEQ ID | AP2 | I | H-2Dd | KRKSIRIQRGPGR | 53935 |
| NO: 2 | | | | AFVTGKIGNMRQ | |
| SEQ ID | AP3 | I | H-2Dd | TLTCGFADLMGYI | 716 |
| NO: 3 | | | | PLVGAPLGGAAR | |
| SEQ ID | AP4 | I | H-2Kd | GLLLVLIYLYGPS | 74070 |
| NO: 4 | | | | LYRRFFSNDCCS | |
| SEQ ID | AP5 | I | H-2Kd | NRFVSIFASGPSQ | 58200 |
| NO: 5 | | | | KIQLVNNNGSWH | |
| SEQ ID | AP6 | I | H-2Kd | KRKSIRIQRGPGR | 53935 |
| NO: 6 | | | | AFVTIGKIGNMR | |
| SEQ ID | AP7 | I | H-2Kd | DTIWALSVKYGVS | 34446 |
| NO: 7 | | | | VQDIMSWNNLSS | |
| SEQ ID | AP8 | I | H-2Ld | LGRLMYDMYPHFM | 75317 |
| NO: 8 | | | | PTNLGPSEKRVW | |
| SEQ ID | AP9 | I | H-2Ld | LLTRILTIPQSLD | 28023 |
| NO: 9 | | | | SWWTSLNFLGGS | |
| SEQ ID | AP10 | II | H-2IAd | ESLKISQAVHAAH | 28676 |
| NO: 10 | | | | AEINEAGREVVG | |
| SEQ ID | AP11 | II | H-2IEd | EQLSSVSSFERFE | 57751 |
| NO: 11 | | | | IFPKESSWPNHN | |
| SEQ ID | AP12 | II | H-2IEd | HPVTIGECPKYVR | 6802 |
| NO: 12 | | | | SAKLRMVTGLRN | |

1) The mouse MHC complex is called H-2
2) There are 3 MHC class I loci: H-2D, H-2K and H-2L
3) There are 2 MHC class II loci: H-2IA and H-2IE
4) Because the genes are polymorphic the alleles are designated with a superscript letter (e.g. H-2D^{d} in Balb/c)
5) Inbred mice have the same allele designation for all loci (e.g. H-2^{d} for Balb/c)
6) Not all strains have all loci (e.g. C57 Black/6 only have H-2D^{b}, H-2K^{b} and H-2IE^{b}). They are null for the other alleles
7) Minimal epitopes are highlighted in BOLD (Class II epitopes are usually longer than class I epitopes)

2. Peptide Conjugation

All peptide syntheses followed standard solid-phase peptide methods. For the specific sequences shown, Fmoc-chemistry was used and the coupling reagent was HBTU. Conjugation of Compound 1 was done on the N-terminal just prior to resin cleavage. By way of example, the conditions used in the conjugation step are:

One to two equivalents of Compound 1 from Synthesis Example 1

Equal equivalent of HBTU

Solvent is DMF

Reaction carried out at r.t. overnight

Purity after resin-cleavage is >80%

The peptide conjugates were purified by prep-HPLC using a C18 column. The mobile phase consists of a suitable gradient of increasing B in A (A=0.05%TFA in H₂O; B=0.05%TFA in CH₃CN). The characterization of peptides and conjugated peptides are shown below:

| SEQ ID | | | RT | Area | MS | MS |
|---|---|---|---|---|---|---|
| NO | ID^{a} | Sequence^{a} | (min)^{b} | %^{b} | Calc'd | Obs'd^{c} |
| SEQ ID | *AP1 | *HSNLNDTT | 11.6 | 90 | 3397.34 | 3396.64 |
| NO: 13 | | YQRTRALVR | | | | |
| | | TGMDPRMC | | | | |
| SEQ ID | AP1 | HSNLNDTTY | 10.6 | 96 | 2937.34 | 2937.89 |
| NO: 1 | | QRTRALVRT | | | | |
| | | GMDPRMC | | | | |
| SEQ ID | *AP2 | *KRKSIRIQ | 11.3 | 75 | 3315.42 | 3316.70 |
| NO: 14 | | RGPGRAFVT | | | | |
| | | GKIGNMRQ | | | | |
| SEQ ID | AP2 | KRKSIRIQR | 11.0 | 95 | 2855.42 | 2855.63 |
| NO: 2 | | GPGRAFVTG | | | | |
| | | KIGNMRQ | | | | |
| SEQ ID | *AP3 | *TLTCGFAD | 15.1 | 83 | 2924.94 | 2925.21 |
| NO: 15 | | LMGYIPLVG | | | | |
| | | APLGGAAR | | | | |
| SEQ ID | AP3 | TLTCGFADL | 16.1 | 84 | 2464.94 | 2465.75 |
| NO: 3 | | MGYIPLVG | | | | |
| | | APLGGAAR | | | | |
| SEQ ID | *AP4 | *GLLLVLIY | 16.8 | 78 | 3373.45 | 3373.01 |
| NO: 16 | | LYGPSLYRR | | | | |
| | | FFSNDCCS | | | | |
| SEQ ID | AP4 | GLLLVLIYL | 14.9 | 86 | 2913.45 | 2914.37 |
| NO: 4 | | YGPSLYRRF | | | | |
| | | FSNDCCS | | | | |
| SEQ ID | *AP5 | *NRFVSIFA | 12.6 | 90 | 3261.13 | 3262.88 |
| NO: 17 | | SGPSQKIQL | | | | |
| | | VNNNGSWH | | | | |
| SEQ ID | APS | NRFVSIFAS | 12.4 | 97 | 2801.13 | 2802.12 |
| NO: 5 | | GPSQKIOLV | | | | |
| | | NNNGSWH | | | | |
| SEQ ID | *AP6 | *KRKSIRIQ | 10.9 | 92 | 3300.45 | 3300.51 |
| NO: 18 | | RGPGRAFVT | | | | |
| | | IGKIGNMR | | | | |
| SEQ ID | AP6 | KRKSIRIQR | 14.5 | 89 | 2840.45 | 2840.33 |
| NO: 6 | | GPGRAFVTI | | | | |
| | | GKIGNMR | | | | |
| SEQ ID | *AP7 | *DTIWALSV | 12.7 | 97 | 3274.17 | 3274.61 |
| NO: 19 | | KYGVSVQD | | | | |
| | | IMSWNNLSS | | | | |
| SEQ ID | AP7 | DTIWALSVK | 12.4 | 95 | 2814.17 | 2814.15 |
| NO: 7 | | YGVSVQDI | | | | |
| | | MSWNNLSS | | | | |
| SEQ ID | *AP8 | *LGRLMYDM | 16.2 | 96 | 3499.60 | 3499.98 |
| NO: 20 | | YPHFMPTN | | | | |
| | | LGPSEKRVW | | | | |
| SEQ ID | AP8 | LGRLMYDMY | 13.7 | 88 | 3039.60 | 3040.02 |
| NO: 8 | | PHFMPTN | | | | |
| | | LGPSEKRVW | | | | |
| SEQ ID | *AP9 | *LLTRILTI | 15.7 | 82 | 3279.26 | 3279.62 |
| NO: 21 | | PQSLDSWWT | | | | |
| | | SLNFLGGS | | | | |
| SEQ ID | AP9 | LLTRILTIP | 16.3 | 90 | 2819.16 | 2819.53 |
| NO: 9 | | QSLDSWWTS | | | | |
| | | LNFLGGS | | | | |
| SEQ ID | *AP10 | *ESLKISQA | 12.5 | 82 | 3075.90 | 3075.52 |
| NO: 22 | | VHAAHAEIN | | | | |
| | | EAGREVVG | | | | |
| SEQ ID | AP10 | ESLKISQAV | 10.9 | 90 | 2615.90 | 2616.26 |
| NO: 10 | | HAAHAEINE | | | | |
| | | AGREVVG | | | | |
| SEQ ID | *AP11 | *EQLSSVSS | 13.3 | 92 | 3442.23 | 3442.82 |
| NO: 23 | | FERFEIFPK | | | | |
| | | ESSWPNHN | | | | |
| SEQ ID | AP11 | EQLSSVSSF | 11.5 | 86 | 2982.23 | 2982.53 |
| NO: 11 | | REFEIFPKE | | | | |
| | | SSWPNHN | | | | |
| SEQ ID | *AP12 | *HPVTIGEC | 13.9 | 92 | 3286.39 | 3286.82 |
| NO: 24 | | PKYVRSAKL | | | | |
| | | RMVTGLRN | | | | |
| SEQ ID | AP12 | HPVTIGECP | 10.3 | 88 | 2826.39 | 2826.03 |
| NO: 12 | | KYVRSAKL | | | | |
| | | RMVTGLRN | | | | |

^{a}(*) denotes peptide conjugate of TLR7 agonist Compound 1 (Synthesis Example 1)
^{b}Analytical HPLC comprised of a C18 column and a mobile phase gradient 0-80% 0.05% TFA/MeCN in 0.05% TFA/H₂O
^{c}Mass spectrometry data was collected on an Applied Biosystem Voyager 1099 (positive polarity)

### BIOLOGICAL EXAMPLES

### Biological Example 1: HEK TLR7 Assay

This example demonstrates that the TLR7 agonist Compound 1 maintains TLR7 agonist activity even after conjugation with an amino acid (conjugates described in Synthesis Examples 2 and 3).

HEK-Blue™ TLR7 cells were purchased from Invivogen (San Diego, Calif.). The following description was taken from the product information sheet.

“HEK-Blue™ hTLR7 cells are designed for studying the stimulation of human TLR7 (hTLR7) by monitoring the activation of NF-kB. HEK-Blue™ hTLR7 cells were obtained by co-transfection of the hTLR7 gene and an optimized secreted embryonic alkaline phosphatase (SEAP) reporter gene into HEK293 cells. The SEAP reporter gene is placed under the control of the IFN-b minimal promoter fused to five NF-kB and AP-1-binding sites. Stimulation with a TLR7 ligand activates NF-kB and AP-1 which induce the production of SEAP, which is detected by the HEK-Blue™ Detection cell culture medium.”

A typical assay protocol involved the following steps:

1. Cells were cultured according to the product information sheet.

2. 10 mM compound stock in DMSO were first diluted to 3 mM and then 3-fold serially diluted using DMSO to afford a 10-pt dilution.

3. 3 μl of the diluted DMSO were added to 57 μl HEK-Blue™ Detection media for a further 20-fold dilution.

4. 10 μl of the diluted compound in assay media were added into 40 μl cell culture (in HEK-Blue™ Detection media) in 384-well plate. Final cell concentration=8,000 cells per well.

5. The plates were incubated at 37° C. in 5% CO2 for 16 h. SEAP was determined using a spectrophotometer at 620-655 nm.

The table below summarizes the results of HEK-TLR7 activity.

| | HEK- |
|---|---|
| | TLR7 EC₅₀ |
| Example | (μM) |
| TLR7 Agonist | 0.1 |
| (Synthesis Example 1, Compound 1) | |
| Peptide Conjugate | 0.1 |
| (Synthesis Example 2, Compound 1-alanine conjugate) | |
| Peptide Conjugate | 0.1 |
| (Synthesis Example 3, Compound 1-lysine conjugate) | |

The HEK-TLR7 data demonstrates that the TLR7 agonist activity of Compound 1 is maintained even after covalent conjugation to amino acids, either at the alpha-position or the side chain (e.g. lysine).

### Biological Example 2: Immunogenicity Studies

This prophetic example describes an experiment that compares peptide conjugates (*AP) in mice to an admix of peptide and a known adjuvant (AP-p), and to a peptide alone (AP).

The T cell stimulatory capacity of peptide-TLR7 conjugates (*AP) is compared to unconjugated peptides alone (AP) or unconjugated peptides admixed with Poly I:C (AP-p) after immunization of Balb/c mice. The 12 different epitopes (AP1-AP12) are combined in two vaccine formulations (6 peptides in each) and used to immunize 6-8 week old female Balb/c mice at the indicated doses (twice i.m. at days 0 and 14). Control animals are immunized with vaccine buffer alone. At day 21 of the study, animals are sacrificed and their spleens harvested and homogenized to obtain single cell suspensions of splenocytes. Splenocytes are re-stimulated ex vivo with medium alone, each individual unconjugated peptide or a pool of all 12 unconjugated peptides. After culture, the cells are stained with fluorescently labeled antibodies to CD3, CD4 and CD8 to identify T cell subsets. Next, fluorescently labeled antibodies specific for IFN-g and TNF-a are used to determine the frequency of T cells responding to each peptide individually and the total pool by flow cytometry.

The vaccine dosing of the mice is summarized below:

| Group | N | Vaccine | Dose (μg) |
|---|---|---|---|
| 1 | 2 | Control | N/A |
| 2 | 5 | AP | 50 |
| 3 | 5 | AP | 16.7 |
| 4 | 5 | AP | 5.6 |
| 5 | 5 | AP-p | 50 |
| 6 | 5 | AP-p | 16.7 |
| 7 | 5 | AP-p | 5.6 |
| 8 | 5 | *AP | 50 |
| 9 | 5 | *AP | 16.7 |
| 10 | 5 | *AP | 5.6 |

### Biological Example 3: HEK TLR7 Activity of Compound 1-peptide Conjugates

This example demonstrates that peptides (20-30 amino acids in length) conjugated to the Compound 1 TLR7 agonist exhibit TLR7 cellular activity. In the absence of Compound 1, the “naked” corresponding peptides do not show measurable TLR7 activities.

| Peptide - | | | |
|---|---|---|---|
| Compound 1 | EC₅₀ | Peptide without | EC₅₀ |
| Conjugate | (micromolar) | Compound 1 | (micromolar) |
| *AP1 | 0.027 | AP1 | >30 |
| *AP2 | 0.20 | AP2 | >30 |
| *AP3 | 0.19 | AP3 | >30 |
| *AP4 | 0.69 | AP4 | >30 |
| *AP5 | 0.15 | AP5 | >30 |
| *AP6 | 0.25 | AP6 | >30 |
| *AP7 | 0.34 | AP7 | >10 |
| *AP8 | 0.48 | AP8 | >30 |
| *AP9 | 0.32 | AP9 | >30 |
| *AP10 | 0.21 | AP10 | >30 |
| *AP11 | 0.30 | AP11 | >30 |
| *AP12 | 0.24 | AP12 | >30 |

*denotes peptide conjugate of TLR7 agonist Compound 1 (Example 1)

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

We claim:

1. A peptide conjugate having the structure of Formula I, or a pharmaceutically acceptable salt thereof, 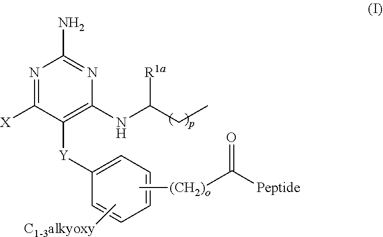
wherein
Peptide is a peptide, wherein (C═O) is attached to (i) the N-terminus of the peptide, or (ii) a side chain of the peptide where the functional group of the side chain to which CO═O is attached is NH₂;
R^{1a} is H or C₁-C₄ alkyl, wherein the alkyl is optionally substituted with one to three substituents selected from the group consisting of —OH, —NH₂, —NHAc, —COOH, —SO₂CH₃, —SCH₃, —OCH₃, 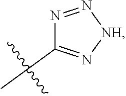 [SMILES: *c1nn[nH]n1]
and A¹;
X is H or C₁-C₄ alkyl, wherein the alkyl is optionally substituted with one to three substituents selected from the group consisting of A², —OH, and —C(CH₃)₂OH;
Y is selected from the group consisting of a bond, —CH₂—, —CF₂, 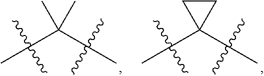
—O—, —S—, —SO₂—, —NH—, and —CH₂CH₂—;
A¹ is selected from the group consisting of 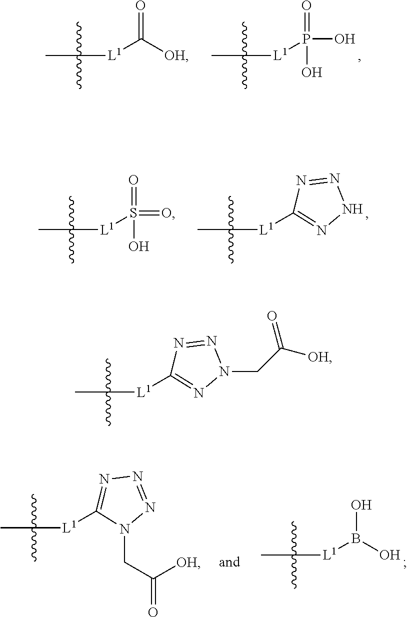
L¹ is selected from the group consisting of a bond, —(CH₂)ₙ—, —C(O)NH(CH₂)ₙ—, 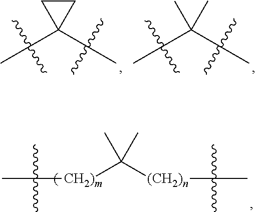
—[O(CH₂CH₂)]ₙ—, —[O(C₁-C₄ alkylene)]-, —[O(CH₂CH₂)]ₙ—OCH₂CH₂CF₂—, —C(O)NHCH₂CH₂—[O(CH₂CH₂)]ₘ—, and —C(O)NHCH₂CH₂—[O(CH₂CH₂)]ₘ—OCH₂CH₂CF₂—;
A² is selected from the group consisting of 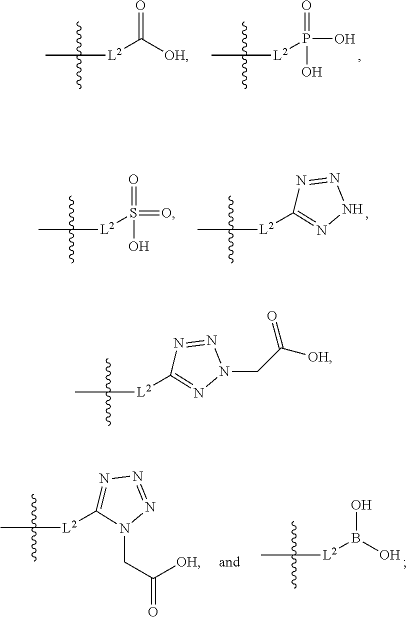
L² is selected from the group consisting of a bond, —(CH₂)ₙ—, —C(O)NH(CH₂)ₙ—, 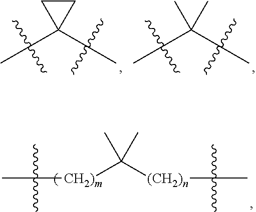
—[O(CH₂CH₂)]ₙ—, —[O(C₁-C₄ alkylene)]-, —[O(CH₂CH₂)]ₘ—, and —C(O)NHCH₂CH₂—[O(CH₂CH₂)]ₘ—OCH₂CH₂CF₂—;
m is an integer from zero to four;
n and p are independently an integer from one to four; and
o is an integer from zero to four.

2. The peptide conjugate of claim 1, or a pharmaceutically acceptable salt thereof, having the structure of Formula (1a) or (2a), or a pharmaceutically acceptable salt thereof, 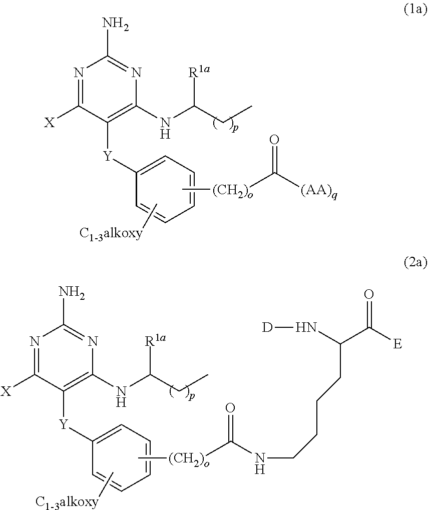
wherein
each AA is independently an amino acid, wherein the (AA)_{q} is a peptide, wherein (C═O) is attached to the N-terminus of the peptide;
q is an integer from eight to forty;
D is H or an amino acid or a peptide comprising 2 to 40 amino acids; and
E is OH or an amino acid or a peptide comprising 2 to 40 amino acids.

3. The peptide conjugate of claim 2, or a pharmaceutically acceptable salt thereof, having the structure of Formula (1b) or (2b), or a pharmaceutically acceptable salt thereof,

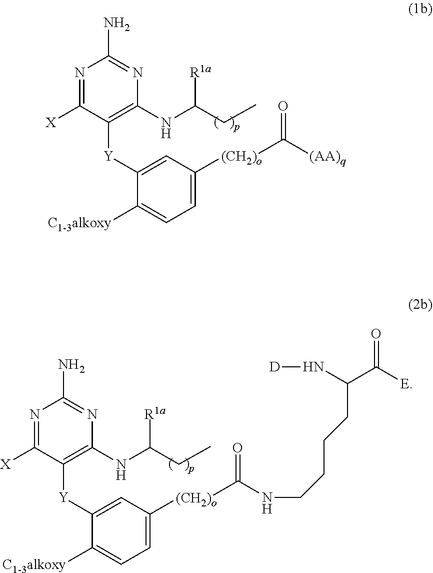

4. The peptide conjugate of any one of claims 1-3, or a pharmaceutically acceptable salt thereof, wherein
R^{1a} is C₁-C₄ alkyl, wherein the alkyl is optionally substituted with one to three substituents selected from the group consisting of —OH, —NH₂, —NHAc, —COOH, —SO₂CH₃, —SCH₃, —OCH₃, 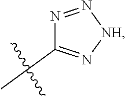 [SMILES: *c1nn[nH]n1]
and A¹;
X is C₁-C₄ alkyl; and
Y is —CH₂—.

5. The peptide conjugate of any one of claims 1-4, or a pharmaceutically acceptable salt thereof, wherein R^{1a} is C₁-C₄ alkyl, and alkyl is substituted with —SO₂CH₃.

6. The peptide conjugate of any one of claims 1-5, or a pharmaceutically acceptable salt thereof, wherein R^{1a} is 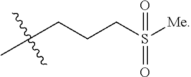 [SMILES: *CCCS(C)(=O)=O]
is

7. The peptide conjugate of any one of claims 1-6, or a pharmaceutically acceptable salt thereof, wherein X is methyl.

8. The peptide conjugate of any one of claims 1-7, or a pharmaceutically acceptable salt thereof, wherein p is three.

9. The peptide conjugate of any one of claims 1-8, or a pharmaceutically acceptable salt thereof, wherein o is one.

10. The peptide conjugate of any one of claims 1-9, or a pharmaceutically acceptable salt thereof, wherein Y is —CH₂—.

11. The peptide conjugate of any one of claims 1-10, or a pharmaceutically acceptable salt thereof, wherein the peptide is an antigen.

12. The peptide conjugate of claim 11, or a pharmaceutically acceptable salt thereof, wherein the antigen is a bacterial or viral antigen.

13. The peptide conjugate of claim 11, or a pharmaceutically acceptable salt thereof, wherein the antigen is an epitope.

14. The peptide conjugate of claim 11, or a pharmaceutically acceptable salt thereof, wherein the antigen is a shared tumor antigen.

15. The peptide conjugate of claim 11, or a pharmaceutically acceptable salt thereof, wherein the antigen is a personalized neoantigen.

16. The peptide conjugate of any one of claims 1-10, or a pharmaceutically acceptable salt thereof, wherein the peptide is a vaccine.

17. A peptide conjugate of any one of claims 1-16, or a pharmaceutically acceptable salt thereof, wherein the peptide is prepared by solid phase synthesis.

18. A pharmaceutical composition comprising the peptide conjugate of any one of claims 1-17, or a pharmaceutically acceptable salt thereof.

19. A method of treating a tumor in a subject in need thereof, comprising administering to the subject in need thereof a peptide conjugate, or a pharmaceutically acceptable salt thereof, of any one of claims 1-17, or a pharmaceutical composition of claim 18.

20. A method of vaccinating a subject in need thereof against a tumor, comprising administering to the subject in need thereof a peptide conjugate, or a pharmaceutically acceptable salt thereof, of any one of claims 1-17, or a pharmaceutical composition of claim 18.

21. The method of claim 19 or 20, wherein the tumor is a solid tumor.

22. A method of making a conjugate of any one of claims 1-17, or a pharmaceutically acceptable salt thereof, comprising the step of conjugating a peptide with a compound of Formula (3a) 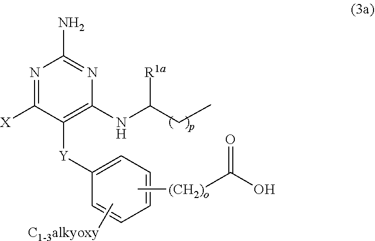
wherein
R^{e}a is H or C₁-C₄ alkyl, wherein the alkyl is optionally substituted with one to three substituents selected from the group consisting of —OZ, —NHZ, —NHAc, —COOZ, —SO₂CH₃, —SCH₃, —OCH₃, 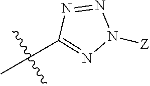
and A¹;
X is H or C₁-C₄ alkyl, wherein the alkyl is optionally substituted with one to three substituents selected from the group consisting of A², —OZ, and —C(CH₃)₂OZ;
Y is selected from the group consisting of a bond, —CH₂—, —CF₂—, 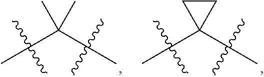
—O—, —S—, —SO₂—, —NH—, and —CH₂CH₂—;
A¹ is selected from the group consisting of 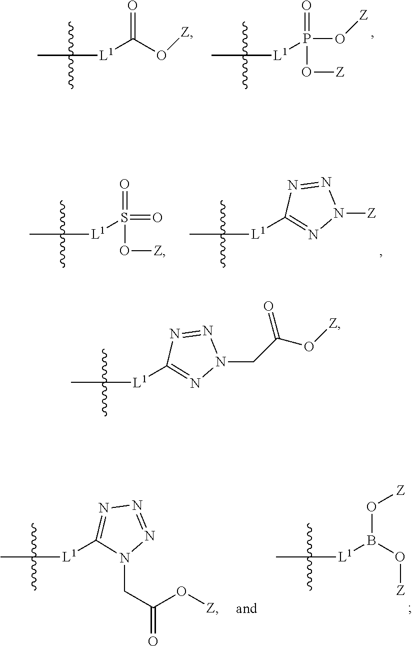
L¹ is selected from the group consisting of a bond, —(CH₂)ₙ—, —C(O)NH(CH₂)ₙ—, 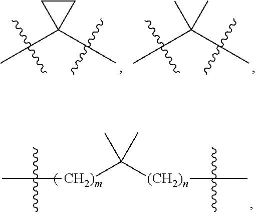
—[O(CH₂CH₂)]ₙ—, —[O(C₁-C₄ alkylene)]-, —[O(CH₂CH₂)]ₙ—OCH₂CH₂CF₂—, —C(O)NHCH₂CH₂-[O(CH₂CH₂)]ₘ—, and —C(O)NHCH₂CH₂—[O(CH₂CH₂)]ₘ—OCH₂CH₂CF₂—;
A² is selected from the group consisting of 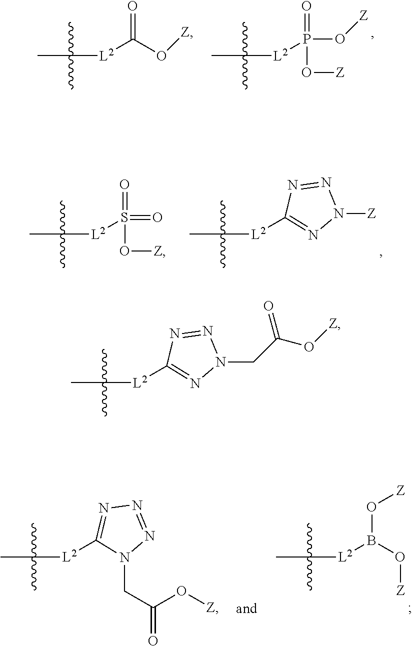
L² is selected from the group consisting of a bond, —(CH₂)ₙ—, —C(O)NH(CH₂)ₙ—, 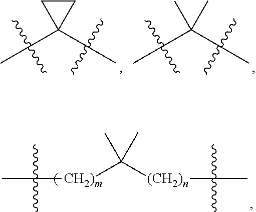
—[O(CH₂CH₂)]ₙ—, —[O(C₁-C₄ alkylene)]-, —[O(CH₂CH₂)]ₙ—OCH₂CH₂CF₂—, —C(O)NHCH₂CH₂-[O(CH₂CH₂)]ₘ—, and —C(O)NHCH₂CH₂—[O(CH₂CH₂)]ₘ—OCH₂CH₂CF₂—;
each Z is independently H or a protecting group;
m is an integer from zero to four;
n and p are independently an integer from one to four; and
o is an integer from zero to four.

23. The method according to claim 22, wherein the peptide is bound to a solid phase.

24. The method according to claim 23, further comprising the step of cleaving the conjugate from the solid phase.

25. A peptide conjugate of any one of claims 1-17, or a pharmaceutically acceptable salt thereof, for use in therapy.

26. A pharmaceutical composition of claim 18, for use in therapy.

27. Use of a peptide conjugate of any one of claims 1-17, or a pharmaceutically acceptable salt thereof, in the manufacture of a medicament.


SEQUENCE LISTING (24 sequences)

SEQ ID NO: 1 - protein, 25 aa, Mus musculus
HSNLNDTTYQRTRALVRTGMDPRMC

SEQ ID NO: 2 - protein, 25 aa, Mus musculus
KRKSIRIQRGPGRAFVTGKIGNMRQ

SEQ ID NO: 3 - protein, 25 aa, Mus musculus
TLTCGFADLMGYIPLVGAPLGGAAR

SEQ ID NO: 4 - protein, 25 aa, Mus musculus
GLLLVLIYLYGPSLYRRFFSNDCCS

SEQ ID NO: 5 - protein, 25 aa, Mus musculus
NRFVSIFASGPSQKIQLVNNNGSWH

SEQ ID NO: 6 - protein, 25 aa, Mus musculus
KRKSIRIQRGPGRAFVTIGKIGNMR

SEQ ID NO: 7 - protein, 25 aa, Mus musculus
DTIWALSVKYGVSVQDIMSWNNLSS

SEQ ID NO: 8 - protein, 25 aa, Mus musculus
LGRLMYDMYPHFMPTNLGPSEKRVW

SEQ ID NO: 9 - protein, 25 aa, Mus musculus
LLTRILTIPQSLDSWWTSLNFLGGS

SEQ ID NO: 10 - protein, 25 aa, Mus musculus
ESLKISQAVHAAHAEINEAGREVVG

SEQ ID NO: 11 - protein, 25 aa, Mus musculus
EQLSSVSSFERFEIFPKESSWPNHN

SEQ ID NO: 12 - protein, 25 aa, Mus musculus
HPVTIGECPKYVRSAKLRMVTGLRN

SEQ ID NO: 13 - protein, 25 aa, Mus musculus
  MISC_FEATURE (1)..(1): conjugated to TLR7 agonist Compound 1
HSNLNDTTYQRTRALVRTGMDPRMC

SEQ ID NO: 14 - protein, 25 aa, Mus musculus
  MISC_FEATURE (1)..(1): conjugated to TLR7 agonist Compound 1
KRKSIRIQRGPGRAFVTGKIGNMRQ

SEQ ID NO: 15 - protein, 25 aa, Mus musculus
  MISC_FEATURE (1)..(1): conjugated to TLR7 agonist Compound 1
TLTCGFADLMGYIPLVGAPLGGAAR

SEQ ID NO: 16 - protein, 25 aa, Mus musculus
  MISC_FEATURE (1)..(1): conjugated to TLR7 agonist Compound 1
GLLLVLIYLYGPSLYRRFFSNDCCS

SEQ ID NO: 17 - protein, 25 aa, Mus musculus
  MISC_FEATURE (1)..(1): conjugated to TLR7 agonist Compound 1
NRFVSIFASGPSQKIQLVNNNGSWH

SEQ ID NO: 18 - protein, 25 aa, Mus musculus
  MISC_FEATURE (1)..(1): conjugated to TLR7 agonist Compound 1
KRKSIRIQRGPGRAFVTIGKIGNMR

SEQ ID NO: 19 - protein, 25 aa, Mus musculus
  MISC_FEATURE (1)..(1): conjugated to TLR7 agonist Compound 1
DTIWALSVKYGVSVQDIMSWNNLSS

SEQ ID NO: 20 - protein, 25 aa, Mus musculus
  MISC_FEATURE (1)..(1): conjugated to TLR7 agonist Compound 1
LGRLMYDMYPHFMPTNLGPSEKRVW

SEQ ID NO: 21 - protein, 25 aa, Mus musculus
  MISC_FEATURE (1)..(1): conjugated to TLR7 agonist Compound 1
LLTRILTIPQSLDSWWTSLNFLGGS

SEQ ID NO: 22 - protein, 25 aa, Mus musculus
  MISC_FEATURE (1)..(1): conjugated to TLR7 agonist Compound 1
ESLKISQAVHAAHAEINEAGREVVG

SEQ ID NO: 23 - protein, 25 aa, Mus musculus
  MISC_FEATURE (1)..(1): conjugated to TLR7 agonist Compound 1
EQLSSVSSFERFEIFPKESSWPNHN

SEQ ID NO: 24 - protein, 25 aa, Mus musculus
  MISC_FEATURE (1)..(1): conjugated to TLR7 agonist Compound 1
HPVTIGECPKYVRSAKLRMVTGLRN